(19) United States
(12) Patent Application Publication
(10) Pub. No.: US 2025/0281672 A1
(43) Pub. Date: Sep. 11, 2025

(54) IDENTIFICATION OF PRO- AND ANTI-INFLAMMATORY LIPIDS RELATING TO IMMUNE RESPONSE TO MEDICAL IMPLANTS

(71) Applicant: William Marsh Rice University, Houston, TX (US)

(72) Inventors: Omid VEISEH, Houston, TX (US); Christian SCHREIB, Houston, TX (US); Maria RUOCCO, Houston, TX (US); Tanguy TERLIER, Houston, TX (US); Sudip MUKHERJEE, Houston, TX (US)

(73) Assignee: William Marsh Rice University, Houston, TX (US)

(21) Appl. No.: 18/293,734

(22) PCT Filed: Jul. 29, 2022
(86) PCT No.: PCT/US2022/074312

§ 371 Date: Jan. 30, 2024

Related U.S. Application Data
(60) Provisional application No. 63/227,437, filed on Jul. 30, 2021.

(51) Int. Cl. A61L 27/28; A61L 27/54; A61L 29/08; A61L 29/16; G01N 33/92

(52) U.S. Cl. CPC A61L 27/28; A61L 27/54; A61L 29/08; A61L 29/16; G01N 33/92; A61L 2300/22; A61L 2420/02

(57) ABSTRACT

The present disclosure is directed to the identification and use of lipids involved in auto-immune reactions and foreign body responses. A. set of phospholipids was observed to preferentially deposit on immune-evasive implants, and these phospholipids upregulated RNA transcripts of anti-inflammatory proteins in murine macrophages. Additionally, a set of fatty acid lipids was observed to preferentially deposit on immunogenic implants, and these fatty acid lipids upregulated RNA transcripts of inflammatory proteins in murine macrophages. Using selected lipids, implantable devices can be engineered to avoid or to attract host immune responses, thereby regulating the length of time the devices are maintained in vivo.

### PRIORITY CLAIM

This application is a national phase application under 35 U.S.C. $371 of International Application No. PCT/US2022/074312, filed Jul. 27, 2022, which claims benefit of priority from U.S. Provisional Application Ser. No. 63/227,437, filed Jul. 30, 2021, the entire contents of which are hereby incorporated by reference.

### STATEMENT REGARDING FEDERALLY SPONSORED RESEARCH

This invention was made with government support under grant no. R01DK-120459 awarded by the National Institutes of Health and under grant no. CBET-1626418 awarded by the National Science Foundation. The government has certain rights in the invention.

### BACKGROUND

### 1. Field of the Disclosure

The present disclosure relates generally to the fields of medicine, immunology and medical devices. More particularly, it relates to the identification and exploitation of immune-modulating lipids that play a role in host responses to implanted materials.

### 2. Background

Foreign body responses (FBR) and fibrosis cause failure in many biomedical devices such as pacemakers, cerebrospinal fluid shunts, coronary stents, urogynecology meshes, breast implants, drug delivery pumps, and biosensors (McKeen, 2014; Saini, 2015; Rolfe et al., 2011; Someya et al., 2016). The fibrotic response to implants has traditionally been understood to be initiated when proteins adsorb to implant surfaces and undergo conformational changes, resulting in the formation of a provisional matrix that allows macrophages and fibroblasts to attach and form a fibrotic capsule (Foggia et al., 2019; Klopfleisch and Jung, 2017; Jenney and Anderson, 2000; Vishwakarma et al., 2016). Yet, many other factors can affect the immune response, including surface texture and implant stiffness (Doloff et al., 2021; Noskovicova et al., 2021). Lipid molecules are known to have potent immunologic effects which can influence inflammation and fibrosis (Figueroa et al., 2002; Chiurchiù et al., 2018), although it is not known if they play a role in the immune response to implants. Specific classes of lipids have even been identified as critical players in anti- and pro-inflammatory pathways in disease pathologies, such as cholesterol in cardiovascular diseases, classical eicosanoids in asthma and multiple sclerosis, and lysophospholipids in rheumatoid arthritis (Chiurchiù et al., 2018; Binder et al., 2016; Hotamisligil, 2017).

### SUMMARY

Thus, in accordance with the present disclosure, there is provided a method of identifying a lipid having immune regulating properties comprising (a) providing;
(i) a surface coated with one or more small molecules that are immune-evasive; or
(ii) an uncoated surface or a surface coated with one or more small molecules that are immune-evoking;
(b) introducing said surface into a living animal; (c) removing said surface after a period of time sufficient for lipids to interact with said surface; and (d) identifying a lipid deposited on the surface of said coating. The surface is a flat surface (e.g., a disc), a curved surface, such a sphere, the interior or exterior of a tube (e.g., a catheter), on a bead, or on a rod. The surface may comprise an organic polymer, such as polydimethylsiloxane (PDMS), or a hydrogel, such as an alginate. The one or more small molecules that are immune-evasive may be Z1-Y12 and/or Z1-Y15. The uncoated surface may comprise an organic polymer, such as polydimethylsiloxane (PDMS) or a hydrogel, such as an alginate. The one or more small molecules that are immune-evoking may be Z1-Y12A.

The period of time in step (c) may be about 24 hours or more, such as 1 day, 2 days, 3, days, 4, days, 5, days, 6, days, 7 days, 10 days, two weeks, three weeks or four weeks. Step (d) may comprise mass spectrometry (e.g., Time of Flight-Secondary Ion Mass Spectrometry (ToF-SIMS),) chromatography (e.g., thin layer chromatography, gas chromatography, high pressure liquid chromatography), nuclear magnetic resonance spectroscopy, or any combination thereof. The animal may be a human or non-human mammal. The surface may be implanted subcutaneously, inserted into a body orifice, implanted intraperitoneally, or implanted into the brain.

Also provided is medical device coated with (a) one or more immune-evasive lipids; or (b) one or more immune-evoking lipids, wherein one or more immune-evasive lipids comprise a phospholipid, or wherein one or more immune-evoking lipids comprise a fatty acid. The phospholipid may be selected from phosphatidyl choline, phosphatidyl ethanolamine, phosphatidyl inositol and/or sphingomyelin. The device may be coated with two or more of said phospholipids. The fatty acid may be selected from the fatty acids of Table 2. The device may comprise two or more of said fatty acids. The medical device may further comprise other immune-evasive modifications including non-fouling hydrophilic polymeric film coatings, semi-permeable hydrogel coatings, surface modification with immunomodulatory proteins, surface topography/geometry modifications, immunomodulating cells, surface charge modifications, and combination thereof.

The medical device may be an implantable device, a cardiac pacemaker, a catheter, a needle injection catheter, a blood clot filter, a vascular transplant, a balloon, a stent transplant, a biliary stent, an intestinal stent, a bronchial stent, an esophageal stent, a ureteral stent, an aneurysm-filling coil or other coil device, a surgical repair mesh, a breast implant, a silicone implant, a transmyocardial revascularization device, a percutaneous myocardial revascularization device, a prosthesis, an artificial organ, an artificial vessel, a tube, an organ replacement part, a fiber, a hollow fiber, a membrane, a textile, a dialyzer, a connecting piece, a sensor, a valve, an endoscope, a filter, or a pump chamber. The medical device may be comprised of an organic polymer, such as polydimethylsiloxane (PDMS), or a hydrogel, such as an alginate. The medical device may have multiple layers of immune-evasive lipids and/or immune-evoking lipids.

Further provided is a method of treating a disease or disorder comprising implanting a medical device as described here into a subject in need thereof. The medical device may be co-delivered with and immune-modulatory small molecule drug or biologic.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1a-d. Fabrication and characterization of immunogenic and immune-evasive PDMS disks. (FIG. 1a) Chemical structures of the different small molecules used for surface functionalization. (FIG. 1b) Carpet plots of the spatial distribution of CN⁻ ion on the functionalized surface via SIMS Image Analysis. These ions are indicative of the Z2A, Z2, and Z1 small molecule surface functionalization. Area presented in 300 μm×300 μm. (FIG. 1c) Disks were removed at 28 days post-implantation and imaged using a stereoscope microscope to detect gross levels of fibrosis (n=5). Scale bar represents 5 mm. (FIG. 1d) Histology of tissue around 28-day implants. Arrow highlights where the tissue interfaced with the implanted PDMS. Scale bar represents 1 mm.

FIGS. 2a-j. Specific lipids deposit on implant surfaces depending on the materials' immunogenicity (FIG. 2a) Post-explant images of PDMS disks after being implanted in mice for 24 hours. Whole disk images were taken on a stereoscope, transmitted and DAPI-stained images were taken on a confocal microscope. Z2A, Z2, and Z1 stand for PDMS functionalized with those molecules. Scale bar in whole disk images represents 1 mm, scale bar in transmitted and DAPI stained images represent 200 μm (FIG. 2b) ToF-SIMS images of a select region of a PDMS explanted disk containing cells. Red highlights total ion intensity, blue highlights the sum of ions associated with lipids in the membranes of cells (C₃H₄NO₂⁻, C₂H₄NO⁻, C₇H₁₇⁻, C₁₂H₂₁⁻, C₁₃H₂₅⁻, C₁₅H₂₇⁻), green highlights the sum of ions associated with the amino acid alanine (C₂HN⁻, C₂H₂N⁻, C₂H₄N⁻, C₂H₂NO₂⁻, C₃H₆NO₂⁻). ECV stands to extracellular vesicle. Scale bar represents 50 μm. (FIG. 2c) ToF-SIMS images of a select region of a PDMS explanted disk without cells. Red highlights total ion intensity. Scale bar represents 50 μm. (FIGS. 2d-f) Comparison of ion intensities associated with proteins (d, C₂H₆N⁻), phospholipids (e, PO₂⁻), and fatty acids (f, C₃H₆O₂⁻) on the PDMS disks implanted for 24 hours (n=4). Ion intensities are normalized to total ion intensity of the spectrum they are from. (FIG. 2g) Heatmap of specific lipid species identified from the ToF-SIMS standard library identified on PDMS, PDMS-Z2, PDMS-Z2A, and PDMS-Z1 disks (n=6). Colors are scaled across columns with max value set to 1 and min value set to 0. Specific lipids associated with each heatmap row can be found in Supplementary Table 1 and 2. Phospholipids are identified from ToF-SIMS reference spectra, fatty acids are identified from LIPID MAPS. (FIG. 2h) Fatty acids found on human IP catheters that were explanted from patients due to failure from fibrosis. Color scale used is the same from FIG. 9g. (FIG. 2i) 28-day nonfunctionalized PDMS explants in C57BL/6 and ApoE knockout mice. ApoE knockout mice are deficient in an essential protein for lipid metabolism. Scale bar represents 5 mm. (FIG. 2j) Histology of tissue around the 28-day ApoE K.O. Scale bar represents 1 mm. Acronyms used are as follows: PDMS-Z2-Y12 (Z2), PDMS-Z2-Y12Analog (Z2A), PDMS-Z1-Y15 (Z1), phosphatidylinositol (PI), phosphatidylethanolamine (PE), phosphatidylcholine (PC), sphingomyelin (SM). Specific species that each column is associated with can be found in Supplementary Table 1.

FIGS. 3a-e. Immune cells localized to 2-week implants display similar transcriptome differences observed to be caused by lipids ex vivo (FIG. 3a) Schematic of single-cell RNA sequencing (scRNA-seq) study performed. PDMS and PDMS-Z2-Y12 (PDMS-Z2) disks were implanted into the IP space of mice for 2 weeks. Cells localized to the implant were extracted and used to scRNA-seq (FIG. 3b) Single cell transcriptome profiles clustered into groups by k-means clustering for cell identification. T/NK refer to T and natural killer cells, D to dendritic cells, and N to neutrophils. (FIG. 3c) UMAP visualization of cells localized to PDMS and PDMS-Z2 pooled together. Cell type of clusters are determined by expression of cell-specific markers. (FIG. 3d) Volcano plots showing changes in gene expression of the identified cell types in the PDMS and PDMS-Z2 samples. (FIG. 3e) Heatmap showing relative expression of various cytokines across the different cell types. A full list can be found at FIG. 11.

FIGS. 4a-c. Lipids modulate the RNA profile in macrophages cultured on PDMS ex vivo. (FIG. 4a) Confocal images of macrophages cultured on PDMS and PDMS-Z2-Y12 stained with NucBlue (blue) and vinculin antibodies (green). Scale bar represents 25 μm. (FIG. 4b) Heatmap comparing transcript levels of select genes from RNAseq of macrophages cultured in the listed conditions. Heatmap normalized by column with z-score. (FIG. 4c) Transcript level comparison plots of Arg1, Il1m, Fcer2a and Fos from RNA-seq of macrophages cultured in the listed conditions.

FIGS. 5a-d. Phosphatidylinositol, phosphatidylethanolamine, phosphatidylcholine, and sphingomyelin follow the same deposition pattern on different implant locations. (FIG. 5a) CAD of PDMS implant used in the brain studies. (FIG. 5b, FIG. 5c) Histology and immunohistochemistry, respectively, of brain tissue around where implants were placed for 2 weeks. Circle shows where implant was. (FIG. 5d) Heatmap of specific lipid species identified from the ToF-SIMS standard library (phospholipids) and LIPID MAPS (fatty acids) identified on PDMS and PDMS-Z1 pillars implanted in mice brains space for 2 weeks (n=5). Colors are scaled across columns. Acronyms used are as follows: PDMS-Z1-Y15 (Z1), phosphatidylinositol (PI), phosphatidylethanolamine (PE), phosphatidylcholine (PC), sphingomyelin (SM).

FIGS. 6a-c. Phosphatidylinositol, phosphatidylethanolamine, phosphatidylcholine, sphingomyelin and fatty acids follow the same deposition pattern on different materials. (FIG. 6a) Histology of tissue around PTFE implants in the subcutaneous space for 28-days. Bars show where measurements were taken for quantification of the fibrotic capsule thickness. Scale bar represents 100 μm. (FIG. 6b) Quantification of thickness of fibrotic capsule that formed on the PTFE subcutaneous implants after 28 days. (n=3) (FIG. 6c) Heatmap of specific lipid species identified from the ToF-SIMS standard library (phospholipids) and LIPID MAPS (fatty acids) identified on PTFE and PTFE-Z2-Y12 disks implanted in the subcutaneous space for 24 hours (n=5). Colors are scaled across columns. Acronyms used are as follows: PTFE-Z2-Y12 (Z2), phosphatidylinositol (PI), phosphatidylethanolamine (PE), phosphatidylcholine (PC), sphingomyelin (SM).

FIGS. 7a-c. Chemical synthesis schematics for (FIG. 7a) Z2-Y12, (FIG. 7b) Z2-Y12Analog, and (FIG. 7c) Z1-Y15.

FIGS. 8a-b. Characterization of coated PDMS disks. (FIG. 8a) Mean value with standard deviation of the intensity of the identifier ion fragment C₃N₃H₅⁻ on pre-implanted disks (n=3). The C₃N₃H₅⁻ ion is present in the fragmentation pattern of each small molecule. Statistics performed: One-Way ANOVA with the Tukey's multiple comparison test (α=0.05). (FIG. 8b) Contact angle measurements of disk surfaces against water drops represented as mean+/−standard deviation (n=5).

FIG. 9. Replicates of DAPI staining of PDMS disks implanted in the mouse IP space for 24 hours. Post-explant images of PDMS disks after being implanted in mice for 24 hours. Images were taken on a confocal microscope. These are replicates for the images presented in FIG. 12a. Scale bars represent 200 μm.

FIGS. 10a-b. Quantitation of lipid deposition on implant surfaces. Lipids deposited on the implants were identified through ION TOF Surface Lab Version 7.1, Spectroscopy Program. (FIG. 10a) Lipids that preferentially deposited on the immune-evasive materials (n=6). Ions indicative of the listed phospholipid classes were compared. (FIG. 10b) Lipids that preferentially deposited on the immunogenic materials (n=6). One-Way ANOVA with the Fisher's multiple comparison test, one star indicates an alpha of 0.05, two stars indicates an alpha of 0.01, and 3 stars indicates an alpha of 0.001. Acronyms used are as follows: PDMS-Z2-Y12 (Z2), PDMS-Z2-Y12Analog (Z2A), PDMS-Z1-Y15 (Z1), phosphatidylinositol (PI), phosphatidylethanolamine (PE), phosphatidylcholine (PC), sphingomyelin (SM), and fatty acids (FA). The central red line represents the median, the top of the box represents the 25th percentile of the samples, the bottom of the box represents the 75th percentile of the samples, and the red dot represents an outlier in the data set.

FIG. 11. scRNA-seq expression level of cytokine and chemokines in different cell types localized to mouse IP space implants. Full heatmap showing relative expression of cytokines across the different cell types from the scRNAseq experiment presented in FIGS. 11a-e.

FIG. 12. Comparison of bulk transcriptional changes in macrophages cultured on uncoated PDMS and PDMS-Z2 measure by RNAseq. Macrophages were isolated from the IP space of mice and cultured on PDMS and PDMS-Z2-Y12 for 18 hours and harvested for bulk RNAseq (n=3).

FIGS. 13a-b. ToF-SIMS analysis suggesting successful spin-coating of lipids onto PDMS disks. (FIG. 13a) Characteristic peaks of fatty acids found on representative PDMS disks coated spun coated with fatty acids. (FIG. 13b) Characteristic peaks of phospholipids found on representative PDMS-Z2 disks spun coated with phospholipids.

### DESCRIPTION OF ILLUSTRATIVE EMBODIMENTS

As discussed above, inflammatory reactions and the fibrotic response cause failure in a multitude of biomedical devices. Methods to mitigate fibrosis have traditionally been focused on engineering the physical and chemical properties of materials to reduce the initial protein deposition and disrupt the maturation of deposited immune cells (Vishwakarma et al., 2016; Bridges et al., 2008; Chen et al., 2005; Susssman et al., 2014). More recently, immunomodulatory triazole small molecules known as Z1-Y15 and Z2-Y12 (referred to here as Z1 and Z2 respectively), have been synthesized that confer anti-FBR properties to materials they are functionalized on without following these traditional approaches (Vegas et al., 2016a; 2016b; Xie et al., 2018; Bose et al., 2020; Ghanta et al., Bochenek et al., 2018). These small molecule surface modifications have successfully mitigated the immune response in mice and non-human primates and one is currently in clinical trials (Vegas et al., 2016a; Bochenek et al., 2018; “Safety & Efficacy of Encapsulated Allogeneic FVIII Cell Therapy in Haemophilia A, 2020). Yet, despite this success, the mechanism by which Z1 and Z2 functions are unknown. Since these molecules were not designed to disrupt protein deposition or immune cell maturation directly, their mechanism of action could potentially be affecting lipid deposition.

Here, the inventors employed time-of-flight secondary ion mass spectroscopy (ToF-SIMS) to resolve profiles of deposited lipids on chemically-functionalized implants and utilize RNA sequencing techniques to determine the effect these lipids have on immune cells. ToF-SIMS is a surface analysis technique that can produce mass spectral images with sub-micron resolution and high chemical sensitivity, making it a powerful technique for profiling the composition of deposited lipids on biomaterial surfaces. To elucidate the role that lipids play in the biomaterial-induced FBR, the inventors performed a series of short and long-term implantation studies, to monitor early lipid adsorption (24 hours) and long-term immune responses and fibrosis outcomes. They compared results of Z2, Z1, and non-functionalized implants, as well as implants functionalized with a structural analog of lead Z2 that the inventors further synthesized to probe the physiochemical versus specific biomolecular interaction contributing to FBR and lipid deposition. These analyses revealed that surface functionalization with small molecules had a dramatic impact on lipid deposition profiles.

The analyses reported here revealed that lipids deposit on biomaterials within 24 hours of implantation and may directly regulate the fibrotic response. Different sets of lipid were observed to preferentially deposit on functionalized compared to control implants. Anti-inflammatory phospholipids were found to be enriched significantly on anti-FBR implants, while fatty acids were enriched on unmodified implants. Additionally, murine macrophages cultured in the presence of lipids exhibited different inflammatory phenotypes depending on whether the macrophages were cultured in the presence of phospholipids or fatty acids. Although protein deposition is traditionally considered the main molecular initiator of the FBR, this study provides new evidence that suggests lipids play this same role as well.

These and other aspects of the disclosure are described in detail below.

### I. ASSAYS

In one embodiment, the disclosure is directed to assays designed to identify lipids that are immune modulatory and that arise in response to implanted materials. The assays employ an implanted material that can evoke or avoid an immune response, followed by analysis of the biomaterials that are found on the material after a period of time sufficient for an immune response, if any, to develop.

### A. Implanted Materials and Implantation

The implanted materials make take any shape but in its simplest sense provide a surface for interaction with the host's immune system. The surface may be flat surface or a curved surface, and can take a variety of more complex forms such a sphere, a tube (e.g., inside or outside of the tube), a bead, a rod, a wire, or even more complex 3-D structures such as medical devices.

In one aspect, the surface is made of a material that is itself immune evoking. In other aspect, the surface is coated with a distinct material that is either immune evoking or immune evasive. The inventors have developed a library of such compounds represented by the following formulae:
a compound of the formula:

A-L-R₁ (I)
wherein:
   A is a polymer
   L is a linker of the formula:

NRₐX₁(CH₂CH₂O)ₒ
wherein:
   Rₐ is hydrogen, alkyl_{(C≤6)}, or substituted alkyl_{(C≤6)};
   o is 2, 3, 4, or 5; and
   X₁ is alkanediyl_{(C≤8)} or substituted alkanediyl_{(C≤8)};
or a linker of the formula:

NR_{b}(CH₂)ₚX₂
wherein:
   R_{b} is hydrogen, alkyl_{(C≤6)}, or substituted alkyl_{(C≤6)};
   p is 1, 2, or 3; and
   X₂ is arenediyl_{(C≤12)} or substituted arenediyl_{(C≤12)};
R₁ is a cycloalkyl_{(C≤12)}; haloaryl_{(C≤12)}; S containing heteroaryl_{(C≤12)}; substituted S-containing heteroaryl_{(C≤12)}; alkyl_{(C≤6)}, haloalkyl_{(C≤6)}, alkenyl_{(C≤6)}, or alkynye_{(C≤6)} substituted aryl_{(C≤12)}; aralkyl_{(C≤12)}; substituted aralkyl_{(C≤12)}; heterocycloalkyl_{(C≤12)}; substituted heterocycloalkyl_{(C≤12)}; 2-pyridinyl; 3-aminophenyl; 4-alkoxy_{(C≤6)} substituted aryl_{(C≤12)}; or a group of the formula:

X₃OR₂
wherein:
   X₃ is alkanediyl_{(C≤8)} or substituted alkanediyl_{(C≤8)};
   R₂ is aryl_{(C≤12)} or substituted aryl_{(C≤12)};
or a pharmaceutically acceptable salt thereof.
   The compound may be further defined as:

A-L-R₁ (I)
wherein:
   A is a polymer
   L is a linker of the formula:

NRₐX₁(CH₂CH₂O)ₘ
wherein:
   Rₐ is hydrogen, alkyl_{(C≤6)}, or substituted alkyl_{(C≤6)};
   m is 2, 3, 4, or 5; and
   X₁ is alkanediyl_{(C≤8)} or substituted alkanediyl_{(C≤8)};
R₁ is a cycloalkyl_{(C≤12)}; haloaryl_{(C≤12)}; S containing heteroaryl_{(C≤12)}; substituted S-containing heteroaryl_{(C≤12)}; alkyl_{(C≤6)}, haloalkyl_{(C≤6)}, alkenyl_{(C≤6)}, or alkynye_{(C≤6)} substituted aryl_{(C≤12)}; 3-aminophenyl; 4-alkoxy_{(C≤6)} substituted aryl_{(C≤12)}; or a group of the formula:

X₃OR₂
wherein:
   X₃ is alkanediyl_{(C≤8)} or substituted alkanediyl_{(C≤8)};
   R₂ is aryl_{(C≤12)} or substituted aryl_{(C≤12)};
or a pharmaceutically acceptable salt thereof.
   The compound may be further defined as:

A-L-R₁ (I)
wherein:
   A is a polymer
   L is a linker of the formula:

NRₐX₁(CH₂CH₂O)ₘ
wherein:
   Rₐ is hydrogen, alkyl_{(C≤6)}, or substituted alkyl_{(C≤6)};
   m is 2, 3, 4, or 5; and
   X₁ is alkanediyl_{(C≤8)} or substituted alkanediyl_{(C≤8)};
or a linker of the formula:

NR_{b}(CH₂)ₙX₂
wherein:
   R_{b} is hydrogen, alkyl_{(C≤6)}, or substituted alkyl_{(C≤6)};
   n is 1, 2, or 3; and
   X₂ is arenediyl_{(C≤12)} or substituted arenediyl_{(C≤12)};
R₁ is a haloaryl_{(C≤12)}; aralkyl_{(C≤12)}; substituted aralkyl_{(C≤12)}; heterocycloalkyl_{(C≤12)}; substituted heterocycloalkyl_{(C≤12)}; 2-pyridinyl; 3-aminophenyl;
or a pharmaceutically acceptable salt thereof.
   The polymer may comprises one or more sugar repeating units. The repeating unit may have the formula:

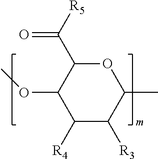

wherein:
   R₃ or R₄ are each independently hydrogen or hydroxy;
   R₅ is a hydroxy, alkoxy_{(C≤8)}, substituted alkoxy_{(C≤8)}, or a covalent bond to the linker; and
   m is a number of repeating units with a molecular weight from about 50,000 Daltons to about 500,000 Daltons.
      The polymer may comprise repeating units of the formula:

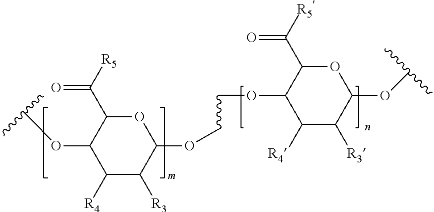

wherein:
   R₃, R₃′, R₄, or R₄, are each independently hydrogen or hydroxy;
   R₅ is a hydroxy, alkoxy_{(C≤8)}, substituted alkoxy_{(C≤8)}, or a covalent bond to the linker;
   R₅′ is a covalent bond to the linker; and
   m and n result in a number of repeating units with a molecular weight from about 50,000 Daltons to about 500,000 Daltons.
      The polymer may be an acrylate polymer, such as a methacrylate polymer.

In particular, the following molecules are useful in the disclosed assays:

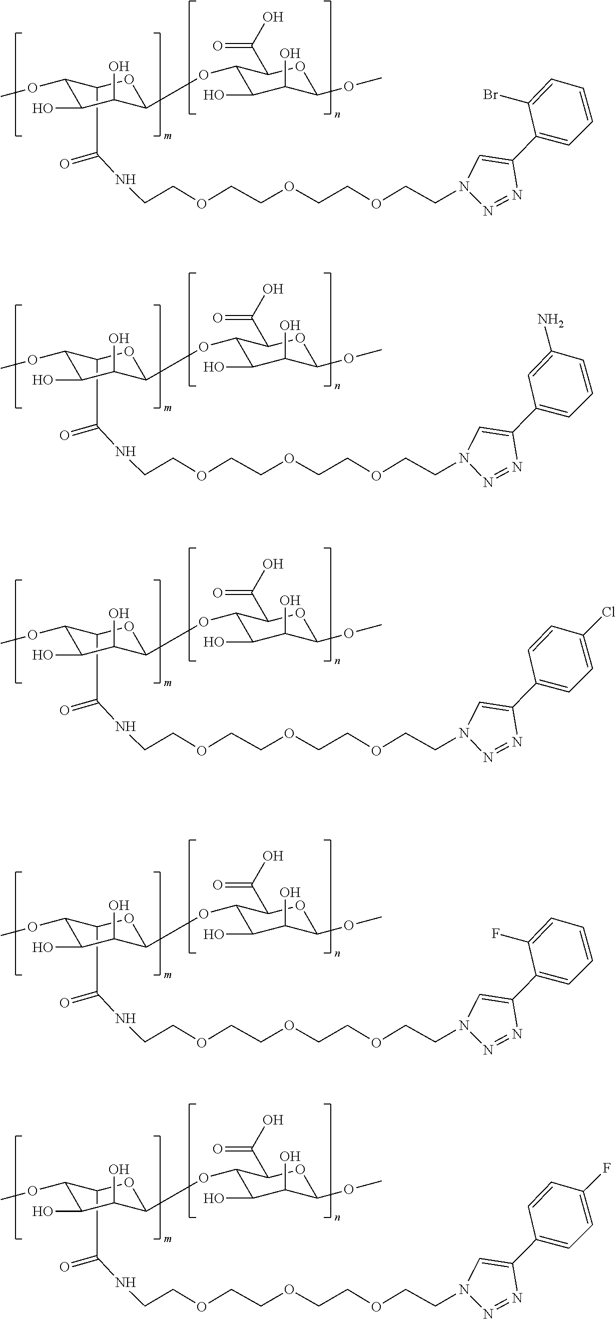

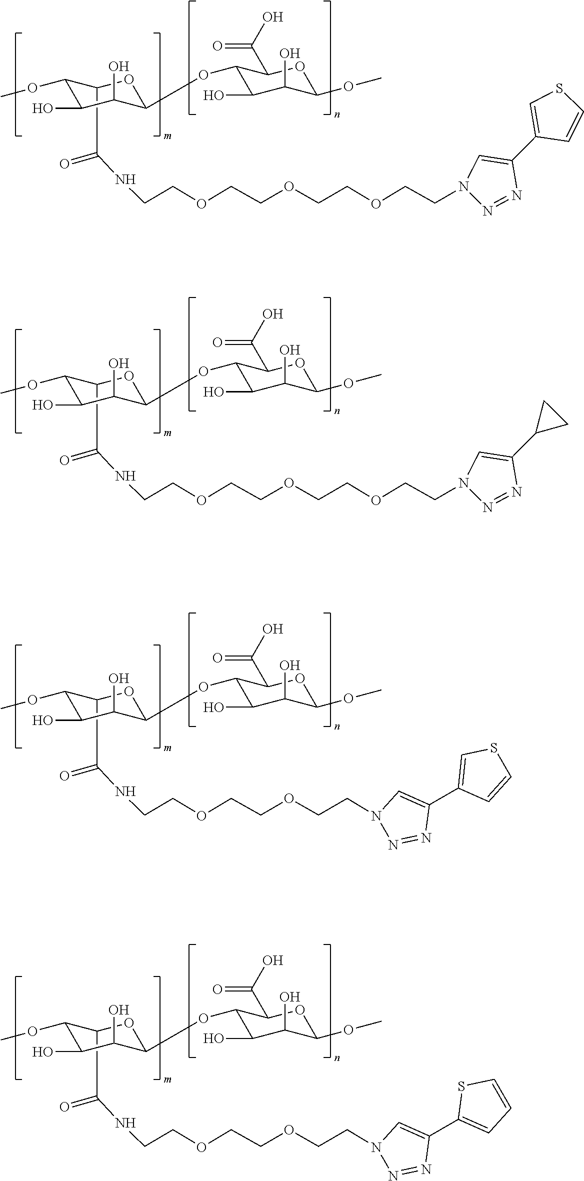

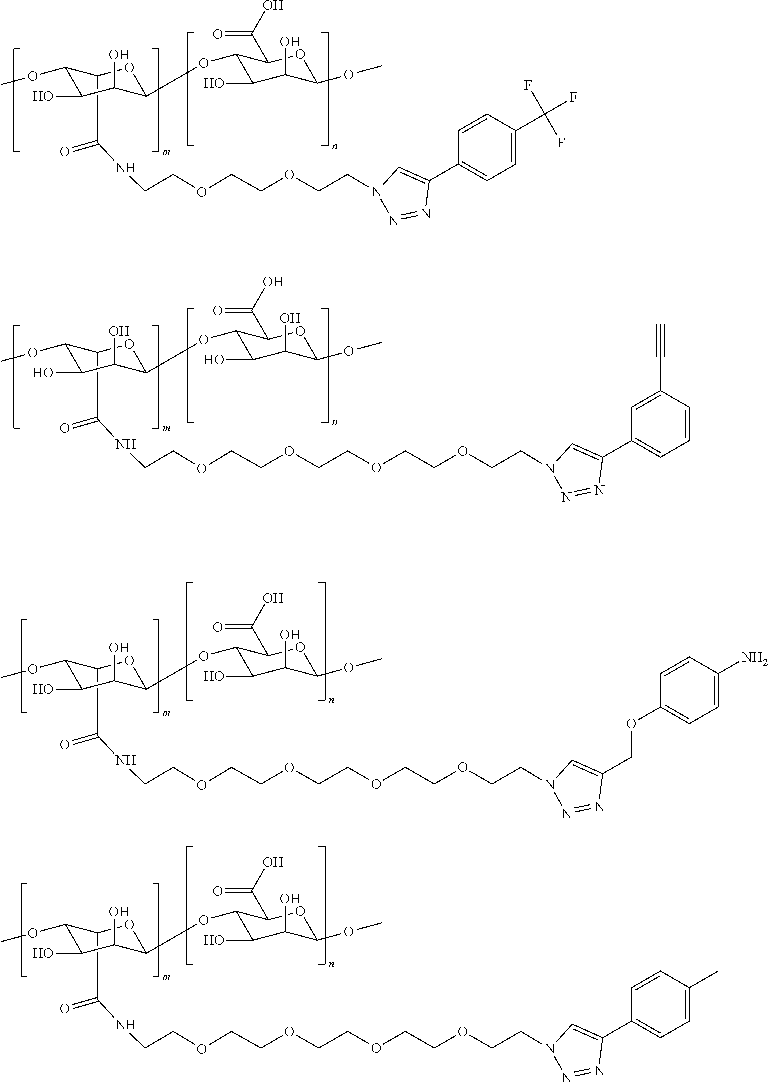

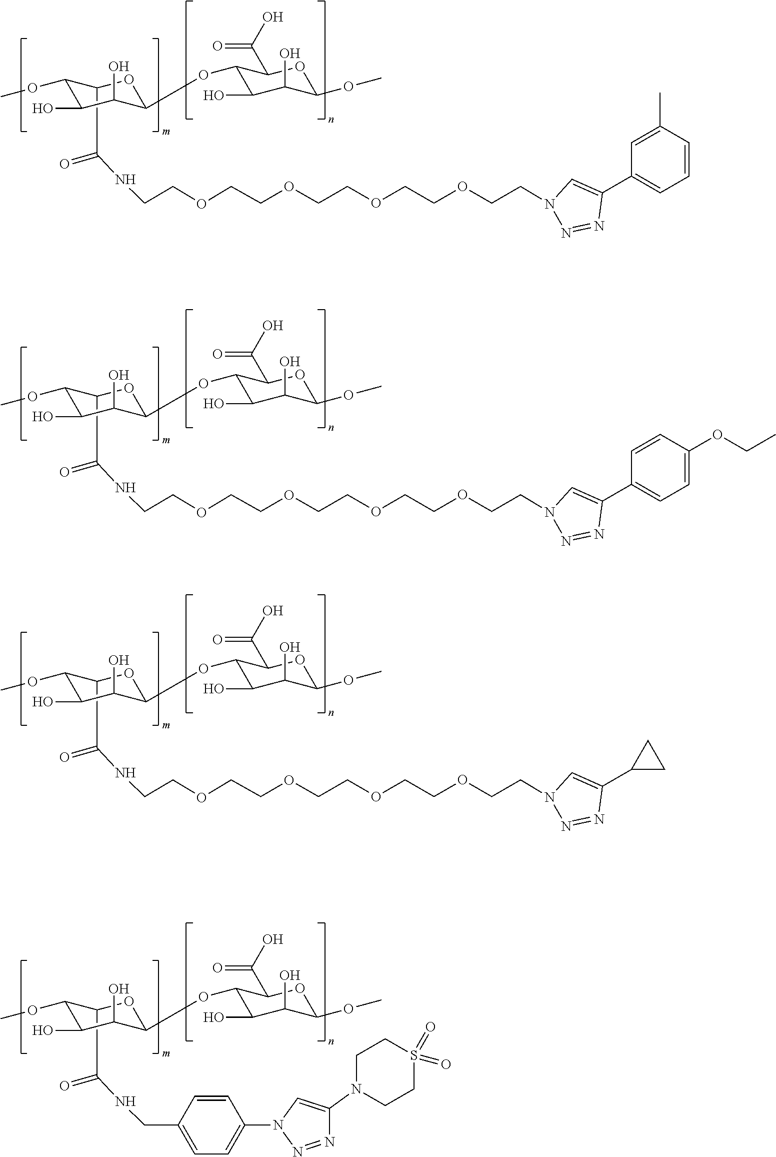

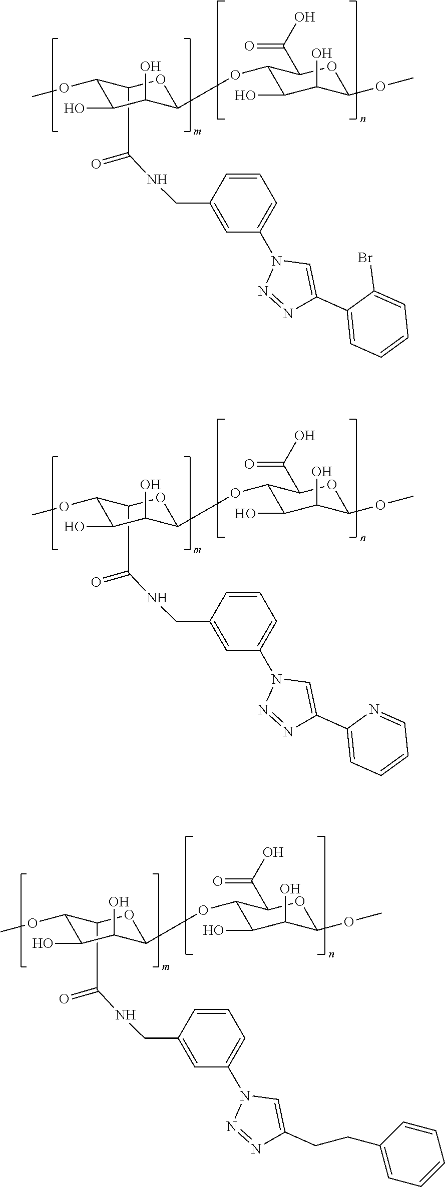

The methods may employ human or non-human animal model hosts. The material/surface can be implanted subcutaneously, intramuscularly or intraperitoneally, implanted into the brain or other organ, or inserted into a body orifice such as mouth, urethra or rectum The implanted/inserted material/surface may be left in situ. for about 24 hours or more, such as 1 day, 2 days, 3, days, 4, days, 5, days, 6, days, 7 days, 10 days, two weeks, three weeks or four weeks.

### B. Detection and Characterization of Biomolecules

Following removal of the material/surface from the host, the surface is assessed for the presence of host biomolecules. The following methods are suitable to such assessments.

### 1. Chromatography

Chromatography is a technique for the separation of a mixture. The mixture is dissolved in a fluid (gas, solvent, water, etc.) called the mobile phase, which carries it through a system (a column, a capillary tube, a plate, or a sheet) on which is fixed a material called the stationary phase. The different constituents of the mixture have different affinities for the stationary phase. The different molecules stay longer or shorter on the stationary phase, depending on their interactions with its surface sites. So, they travel at different apparent velocities in the mobile fluid, causing them to separate. The separation is based on the differential partitioning between the mobile and the stationary phases. Subtle differences in a compound's partition coefficient result in differential retention on the stationary phase and thus affect the separation.

Chromatography may be preparative or analytical. The purpose of preparative chromatography is to separate the components of a mixture for later use and is thus a form of purification. Analytical chromatography is done normally with smaller amounts of material and is for establishing the presence or measuring the relative proportions of analytes in a mixture. The two are not mutually exclusive.

Various types of chromatography are used such as planar chromatography, paper chromatography, thin-layer chromatography, gas chromatography, liquid chromatography, supercritical fluid chromatography, ion exchange chromatography, size exclusion chromatography, reverse phase chromatography, hydrophobic interaction chromatography, hydrodynamic chromatography, two-dimensional chromatography, fast protein liquid chromatography, pyrolysis gas chromatography and countercurrent chromatography.

### 2. Mass Spectrometry

Mass spectrometry (MS) is an analytical technique that is used to measure the mass-to-charge ratio of ions. The results are typically presented as a mass spectrum, a plot of intensity as a function of the mass-to-charge ratio. Mass spectrometry is used in many different fields and is applied to pure samples as well as complex mixtures.

A mass spectrum is a plot of the ion signal as a function of the mass-to-charge ratio. These spectra are used to determine the elemental or isotopic signature of a sample, the masses of particles and of molecules, and to elucidate the chemical identity or structure of molecules and other chemical compounds.

In a typical MS procedure, a sample, which may be solid, liquid, or gaseous, is ionized, for example by bombarding it with a beam of electrons. This may cause some of the sample's molecules to break up into positively charged fragments or simply become positively charged without fragmenting. These ions (fragments) are then separated according to their mass-to-charge ratio, for example by accelerating them and subjecting them to an electric or magnetic field: ions of the same mass-to-charge ratio will undergo the same amount of deflection. The ions are detected by a mechanism capable of detecting charged particles, such as an electron multiplier. Results are displayed as spectra of the signal intensity of detected ions as a function of the mass-to-charge ratio. The atoms or molecules in the sample can be identified by correlating known masses (e.g., an entire molecule) to the identified masses or through a characteristic fragmentation pattern.

Time-of-flight (ToF) mass spectrometry is a method of mass spectrometry in which an ion's mass-to-charge ratio is determined by a time of flight measurement. Ions are accelerated by an electric field of known strength. This acceleration results in an ion having the same kinetic energy as any other ion that has the same charge. The velocity of the ion depends on the mass-to-charge ratio (heavier ions of the same charge reach lower speeds, although ions with higher charge will also increase in velocity). The time that it subsequently takes for the ion to reach a detector at a known distance is measured. This time will depend on the velocity of the ion, and therefore is a measure of its mass-to-charge ratio. From this ratio and known experimental parameters, one can identify the ion.

A particular form of ToF mass spectrometry employs secondary-ion mass spectrometry (SIMS). SIMS is a technique used to analyze the composition of solid surfaces and thin films by sputtering the surface of the specimen with a focused primary ion beam and collecting and analyzing ejected secondary ions. The mass/charge ratios of these secondary ions are measured with a mass spectrometer to determine the elemental, isotopic, or molecular composition of the surface to a depth of 1 to 2 nm. Due to the large variation in ionization probabilities among elements sputtered from different materials, comparison against well-calibrated standards is necessary to achieve accurate quantitative results. SIMS is the most sensitive surface analysis technique, with elemental detection limits ranging from parts per million to parts per billion.

### 3. Nuclear Magnetic Resonance Spectroscopy

Nuclear magnetic resonance spectroscopy, most commonly known as NMR spectroscopy or magnetic resonance spectroscopy (MRS), is a spectroscopic technique to observe local magnetic fields around atomic nuclei. The sample is placed in a magnetic field and the NMR signal is produced by excitation of the nuclei sample with radio waves into nuclear magnetic resonance, which is detected with sensitive radio receivers. The intramolecular magnetic field around an atom in a molecule changes the resonance frequency, thus giving access to details of the electronic structure of a molecule and its individual functional groups. The principle of NMR usually involves three sequential steps. First, there is an alignment (polarization) of the magnetic nuclear spins in an applied, constant magnetic field B₀. Second, the alignment is perturbed by a weak oscillating magnetic field, usually referred to as a radio frequency (RF) pulse. And third, there is detection and analysis of the electromagnetic waves emitted by the nuclei of the sample as a result of this perturbation.

As the fields are unique or highly characteristic to individual compounds, in modern organic chemistry practice, NMR spectroscopy is the definitive method to identify monomolecular organic compounds. Biochemists use NMR to identify proteins and other complex molecules. Besides identification, NMR spectroscopy provides detailed information about the structure, dynamics, reaction state, and chemical environment of molecules. The most common types of NMR are proton and carbon-13 NMR spectroscopy, but it is applicable to any kind of sample that contains nuclei possessing spin.

NMR spectra are unique, well-resolved, analytically tractable and often highly predictable for small molecules. Different functional groups are obviously distinguishable, and identical functional groups with differing neighboring substituents still give distinguishable signals. NMR has largely replaced traditional wet chemistry tests such as color reagents or typical chromatography for identification. A disadvantage is that a relatively large amount, 2-50 mg, of a purified substance is required, although it may be recovered through a workup. Preferably, the sample should be dissolved in a solvent, because NMR analysis of solids requires a dedicated magic angle spinning machine and may not give equally well-resolved spectra. The timescale of NMR is relatively long, and thus it is not suitable for observing fast phenomena, producing only an averaged spectrum. Although large amounts of impurities do show on an NMR spectrum, better methods exist for detecting impurities, as NMR is inherently not very sensitive—though at higher frequencies, sensitivity is higher.

Correlation spectroscopy is a development of ordinary NMR. In two-dimensional NMR, the emission is centered around a single frequency, and correlated resonances are observed. This allows identifying the neighboring substituents of the observed functional group, allowing unambiguous identification of the resonances. There are also more complex 3D and 4D methods and a variety of methods designed to suppress or amplify particular types of resonances. In nuclear Overhauser effect (NOE) spectroscopy, the relaxation of the resonances is observed. As NOE depends on the proximity of the nuclei, quantifying the NOE for each nucleus allows for construction of a three-dimensional model of the molecule.

### II. LIPIDS AND LIPID PREPARATION

A described herein, the inventors have identified a number of immune modulatory lipids, which are described herein. These lipids can be used to coated or impregnate implantable devices to elicit the desire immune response from a subject receiving the device, i.e., to protect the device from immune attack by the host, or to induce an immune response that will control the modulated of the device including the option of eventual destruction.

Immunoprotective/immune evasive lipids include phospholipids such as phosphatidyl choline, phosphatidyl ethanolamine, phosphatidyl inositol and/or sphingomyelin. Immune-evoking lipids include fatty acids, such as those listed in Table 2.

Lipids will either be purified from natural sources, synthesized, or purchased. Purification of lipids from a natural source includes a grinding method (such as ball milling), followed by solvent extraction, followed by analysis to confirm the identity of the lipid (such as mass spec). The exact process of the synthesis of a lipid will change depending on the lipid species, but generally involved a multi-step reaction with validation steps via HPLC, mass spec, and NMR.

### III. IMPLANTABLE DEVICES AND MATERIALS

The device described herein may take any suitable shape or morphology. For example, the present device may be a sphere, spheroid, tube, cord, string, ellipsoid, disk, cylinder, sheet, torus, cube, stadiumoid, cone, pyramid, triangle, rectangle, square, or rod. The device may comprise a curved or flat section. The device may be prepared through the use of a mold, resulting in a custom shape. In specific embodiments, the device is in a sphere confirmation or disc confirmation.

In the case of in vitro or in vivo assays for identifying lipid immune modulatory lipid species, the surface implantable material be non-functional except for the present of the lipid. However, for therapeutic purposes, the device may provide a variety of other functions, such as biosensing, drug delivery, an artificial organ, biostimulation, The device may vary in size, depending, for example, on the use or site of implantation.

For example, the device may have a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. The device may have a section or region with a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. The device may have a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller. The device may have a section or region with a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller.

The device may comprise pores to permit passage of an object, such as a small molecule (e.g., nutrients or waste), a protein, or a nucleic acid. For example, pores may be greater than 0.1 nm and less than 10 μm. In some aspects, the device comprises pores with a size range of 0.1 μm to 10 μm, 0.1 μm to 9 μm, 0.1 μm to 8 μm, 0.1 μm to 7 μm, 0.1 μm to 6 μm, 0.1 μm to 5 μm, 0.1 μm to 4 μm, 0.1 μm to 3 μm, 0.1 μm to 2 μm.

A device described herein may comprise a chemical modification in or on any enclosed material. Exemplary chemical modifications include small molecules, peptides, proteins, nucleic acids, lipids, or oligosaccharides. The device may comprise at least 0.5%, 1%, 2%, 3%, 4%, 5%, 7.5%, 10%, 15%, 20%, 30%, 40%, 50%, 60%, 70%, 80% or more of a material that is chemically modified, e.g., with a chemical modification described herein. The device may be partially coated with a chemical modification, e.g., at least about 5%, 10%, 15%, 20%, 25%, 30%, 35%, 40%, 45%, 50%, 55%, 60%, 65%, 70%, 75%, 80%, 85%, 90%, 95%, 99%, or 99.9% coated with a chemical modification.

In some aspects, the present device is formulated such that the duration of release of the therapeutic and/or diagnostic agent is tunable. For example, the device may be configured in a certain manner to release a specific amount of a therapeutic and/or diagnostic agent over time, e.g., in a sustained or controlled manner. In some embodiments, the device is chemically modified with a specific density of modifications. The specific density of chemical modifications may be described as the average number of attached chemical modifications per given area. For example, the density of a chemical modification on or in the present device may be 0.01, 0.1, 0.5, 1, 5, 10, 15, 20, 50, 75, 100, 200, 400, 500, 750, 1,000, 2,500, or 5,000 chemical modifications per square μm or square mm.

The device may be formulated or configured for implantation in any organ, tissue, cell, or part of a subject. For example, the device may be implanted or disposed into the intraperitoneal space of a subject. The device may be implanted in or disposed on a tumor or other growth in a subject, or be implanted in or disposed about 0.1 mm, 0.5 mm, 1 mm, 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, 1 cm, 5, cm, 10 cm, or further from a tumor or other growth in a subject. The device may be configured for implantation, or implanted, or disposed on or in the skin, a mucosal surface, a body cavity, the central nervous system (e.g., the brain or spinal cord), an organ (e.g., the heart, eye, liver, kidney, spleen, lung, ovary, breast, uterus), the lymphatic system, vasculature, oral cavity, nasal cavity, gastrointestinal tract, bone, muscle, adipose tissue, skin, or other area.

The present device may be formulated for use for any period of time. For example, the present device may be used for 1 hour, 2 hours, 4 hours, 6 hours, 12 hours, 1 day, 36 hours, 2 days, 3 days, 4 days, 5 days, 6 days, 1 week, 2 weeks, 3 weeks, 4 weeks, 5 weeks, 6 weeks, 2 months, 3 months, 4 months, 5 months, 6 months, 1 year, or longer. The present device can be configured for limited exposure (e.g., less than 2 days, e.g., less than 2 days, 1 day, 24 hours, 20 hours, 16 hours, 12 hours, 10 hours, 8 hours, 6 hours, 5 hours, 4 hours, 3 hours, 2 hours, 1 hour or less). The present device can be configured for prolonged exposure (e.g., at least 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more). The present device can be configured for permanent exposure (e.g., at least 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more).

The disclosed devices may be formed from a biocompatible, hydrogel-forming polymer encapsulating the cells to be transplanted. Examples of materials which can be used to form a suitable hydrogel include polysaccharides such as alginate, collagen, chitosan, sodium cellulose sulfate, gelatin and agarose, water soluble polyacrylates, polyphosphazines, poly(acrylic acids), poly(methacrylic acids), poly(alkylene oxides), poly(vinyl acetate), polyvinylpyrrolidone (PVP), and copolymers and blends of each. See, for example, U.S. Pat. Nos. 5,709,854, 6,129,761, and 6,858,229.

In general, these polymers are at least partially soluble in aqueous solutions, such as water, buffered salt solutions, or aqueous alcohol solutions, that have charged side groups, or a monovalent ionic salt thereof. Examples of polymers with acidic side groups that can be reacted with cations are poly(phosphazenes), poly(acrylic acids), poly(methacrylic acids), poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups and sulfonic acid groups.

Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

In particular embodiments, the biocompatible, hydrogel-forming polymer encapsulating the cells is an alginate. Alginates are a family of unbranched anionic polysaccharides derived primarily from brown algae which occur extracellularly and intracellularly at approximately 20% to 40% of the dry weight. The 1,4-linked α-1-guluronate (G) and β-d-mannuronate (M) are arranged in homopolymeric (GGG blocks and MMM blocks) or heteropolymeric block structures (MGM blocks). Cell walls of brown algae also contain 5% to 20% of fucoidan, a branched polysaccharide sulphate ester with I-fucose four-sulfate blocks as the major component. Commercial alginates are often extracted from algae washed ashore, and their properties depend on the harvesting and extraction processes.

Alginate forms a gel in the presence of divalent cations via ionic crosslinking. Although the properties of the hydrogel can be controlled to some degree through changes in the alginate precursor (molecular weight, composition, and macromer concentration), alginate does not degrade, but rather dissolves when the divalent cations are replaced by monovalent ions. In addition, alginate does not promote cell interactions.

Polyethylene glycol (PEG) has been the most widely used synthetic polymer to create macromers for cell encapsulation. A number of studies have used poly(ethylene glycol) di(meth)acrylate to encapsulate a variety of cells. Biodegradable PEG hydrogels can be been prepared from triblock copolymers of poly(α-hydroxy esters)-b-poly (ethylene glycol)-b-poly(α-hydroxy esters) endcapped with (meth)acrylate functional groups to enable crosslinking. PLA and poly(8-caprolactone) (PCL) have been the most commonly used poly(α-hydroxy esters) in creating biodegradable PEG macromers for cell encapsulation. The degradation profile and rate are controlled through the length of the degradable block and the chemistry. The ester bonds may also degrade by esterases present in serum, which accelerates degradation.

Biodegradable PEG hydrogels can also be fabricated from precursors of PEG-bis-[2-acryloyloxy propanoate]. As an alternative to linear PEG macromers, PEG-based dendrimers of poly(glycerol-succinic acid)-PEG, which contain multiple reactive vinyl groups per PEG molecule, can be used. An attractive feature of these materials is the ability to control the degree of branching, which consequently affects the overall structural properties of the hydrogel and its degradation. Degradation will occur through the ester linkages present in the dendrimer backbone.

The biocompatible, hydrogel-forming polymer can contain polyphosphoesters or polyphosphates where the phosphoester linkage is susceptible to hydrolytic degradation resulting in the release of phosphate. For example, a phosphoester can be incorporated into the backbone of a crosslinkable PEG macromer, poly(ethylene glycol)-di-[ethylphosphatidyl (ethylene glycol) methacrylate](PhosPEG-dMA), to form a biodegradable hydrogel. The addition of alkaline phosphatase, an ECM component synthesized by bone cells, enhances degradation. The degradation product, phosphoric acid, reacts with calcium ions in the medium to produce insoluble calcium phosphate inducing autocalcification within the hydrogel. Poly(6-aminoethyl propylene phosphate), a polyphosphoester, can be modified with methacrylates to create multivinyl macromers where the degradation rate was controlled by the degree of derivitization of the polyphosphoester polymer.

Polyphosphazenes are polymers with backbones consisting of nitrogen and phosphorous separated by alternating single and double bonds. Each phosphorous atom is covalently bonded to two side chains. The polyphosphazenes suitable for cross-linking have a majority of side chain groups which are acidic and capable of forming salt bridges with di- or trivalent cations. Examples of preferred acidic side groups are carboxylic acid groups and sulfonic acid groups. Hydrolytically stable polyphosphazenes are formed of monomers having carboxylic acid side groups that are crosslinked by divalent or trivalent cations such as Ca²⁺ or Al³⁺. Polymers can be synthesized that degrade by hydrolysis by incorporating monomers having imidazole, amino acid ester, or glycerol side groups. Bioerodible polyphosphazines have at least two differing types of side chains, acidic side groups capable of forming salt bridges with multivalent cations, and side groups that hydrolyze under in vivo conditions, e.g., imidazole groups, amino acid esters, glycerol and glucosyl. Hydrolysis of the side chain results in erosion of the polymer. Examples of hydrolyzing side chains are unsubstituted and substituted imidizoles and amino acid esters in which the group is bonded to the phosphorous atom through an amino linkage (polyphosphazene polymers in which both R groups are attached in this manner are known as polyaminophosphazenes). For polyimidazolephosphazenes, some of the “R” groups on the polyphosphazene backbone are imidazole rings, attached to phosphorous in the backbone through a ring nitrogen atom.

Organosilicon compounds are organometallic compounds containing carbon-silicon bonds. Organosilicon chemistry is the corresponding science of their preparation and properties. Most organosilicon compounds are similar to the ordinary organic compounds, being colorless, flammable, hydrophobic, and stable to air. In most organosilicon compounds, Si is tetravalent with tetrahedral molecular geometry. Si—O bonds are much stronger (809 kJ/mol compared to 538 kJ/mol) than a typical C—O single bond. The favorable formation of Si—O bonds drives many organic reactions such as the Brook rearrangement and Peterson olefination. The Si—O bond is even stronger than that of the Si—F bond, even though F is more electronegative than O.

Polydimethylsiloxane (PDMS), also known as dimethylpolysiloxane or dimethicone, is an example of a polymeric organosilicon compound. PDMS is the most widely used silicon-based organic polymer due to its versatility and properties leading to many applications. It is particularly known for its unusual rheological (or flow) properties. PDMS is optically clear and, in general, inert, non-toxic, and non-flammable. It is one of several types of silicone oil (polymerized siloxane). Its applications range from contact lenses and medical devices to elastomers; it is also present in shampoos (as it makes hair shiny and slippery), food (antifoaming agent), caulking, lubricants and heat-resistant tiles.

PDMS is viscoelastic, meaning that at long flow times (or high temperatures), it acts like a viscous liquid, similar to honey. However, at short flow times (or low temperatures), it acts like an elastic solid, similar to rubber. Viscoelasticity is a form of nonlinear elasticity that is common amongst noncrystalline polymers. The loading and unloading of a stress-strain curve for PDMS do not coincide; rather, the amount of stress will vary based on the degree of strain, and the general rule is that increasing strain will result in greater stiffness. When the load itself is removed, the strain is slowly recovered (rather than instantaneously). This time-dependent elastic deformation results from the long-chains of the polymer. But the process that is described above is only relevant when cross-linking is present; when it is not, the polymer PDMS cannot shift back to the original state even when the load is removed, resulting in a permanent deformation. However, permanent deformation is rarely seen in PDMS, since it is almost always cured with a cross-linking agent.

If some PDMS is left on a surface overnight (long flow time), it will flow to cover the surface and mold to any surface imperfections. However, if the same PDMS is poured into a spherical mold and allowed to cure (short flow time), it will bounce like a rubber ball. The mechanical properties of PDMS enable this polymer to conform to a diverse variety of surfaces. Since these properties are affected by a variety of factors, this unique polymer is relatively easy to tune. This enables PDMS to become a good substrate that can easily be integrated into a variety of microfluidic and microelectromechanical systems. Specifically, the determination of mechanical properties can be decided before PDMS is cured; the uncured version allows the user to capitalize on myriad opportunities for achieving a desirable elastomer. Generally, the cross-linked cured version of PDMS resembles rubber in a solidified form. It is widely known to be easily stretched, bent, compressed in all directions. Depending on the application and field, the user is able to tune the properties based on what is demanded.

Overall PDMS has a low elastic modulus which enables it to be easily deformed and results in the behavior of a rubber. Viscoelastic properties of PDMS can be more precisely measured using dynamic mechanical analysis. This method requires determination of the material's flow characteristics over a wide range of temperatures, flow rates, and deformations. Because of PDMS's chemical stability, it is often used as a calibration fluid for this type of experiment.

In some embodiments, the implantable devices are medical devices, such as a cardiac pacemaker, a catheter, a needle injection catheter, a blood clot filter, a vascular transplant, a balloon, a stent transplant, a biliary stent, an intestinal stent, a bronchial stent, an esophageal stent, a ureteral stent, an aneurysm-filling coil or other coil device, a surgical repair mesh, a breast implant, a silicone implant, PDMS, a transmyocardial revascularization device, a percutaneous myocardial revasculariza-tion device, a prosthesis, an organ, a vessel, an aorta, a heart valve, a tube, an organ replacement part, an implant, a fiber, a hollow fiber, a membrane, a sensor, a valve, an endoscope, a filter, a pump chamber, or another medical device intended to have hemocompatible properties or used in cancer, diabetes, ischemia, infectious disease, hemophilia, stroke, blood disorder, or a cytokine therapy.

In some embodiments, the implantable materials are coated with immunomodulatory small molecules and/or lipids. Any combination of the small molecules and/or lipids will be used. Molecules can be patterned onto the surface of the materials by applying a mask to the materials prior to plasma cleaning in the coating process.

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Z2-Y12 synthesis. 4-Iodobenzylamide (1 equiv., 5 g, 18.55 mmol) was added in a 50 mL round bottom flask containing 24 mL of methanol followed by sequential addition of triethylamine (2.4 equiv., 4.5 g, 44.52 mmol), sodium azide (2 equiv., 2.41 g, 37.10 mmol), MilliQ water (6 mL), copper iodide (0.15 equiv., 529.86 mg, 2.78 mmol), sodium ascorbate (0.1 equiv., 367.44, 0.1 eq, 3.71 mmol), 2.29 ml trans-N—N′-dimethylcyclohexane-1,2-diamine (0.2 equiv., 527.65 mg, 3.71 mmol). The mixture was evacuated and flushed with argon 3 times and 2-(2-propynyloxy) tetrahydropyran (1 equiv., 2.6 g, 18.55 mmol) was added. The reaction was stirred for 5 mins at room temperature followed by overnight stirring at 55° C. The reaction mixture was allowed to cool to room temperature and filtered over Cellite. The solvent was removed under reduced pressure and the crude reaction was then purified by column chromatography with dichloromethane:ultra (22% MeOH in DCM with 3% NH40H) mixture ranging from 0% to 40% ultra on a 120 g ISCO silica column. The compound was characterized by mass spectroscopy and NMR to confirm the mass and purity.

Z1-Y15 synthesis. 4-Propargylthiomorpholine 1,1-dioxide (1 equiv., 2 g, 11.54 mmol) was added to tris(benzyltriazlylmethyl)amine (TBTA) (0.25 equiv., 1.5 g, 2.83 mmol), triethylamine (0.25 equiv., 292 mg, 2.13 mmol), and copper iodide (0.1 eq, 220 mg, 1.16 mmol) in 5:1 Methanol and MilliQ water (75 mL total). The solution and was purged with argon for 10 min, 3 times. The mixture was then cooled to 0° C. using an ice bath. 11-azido-3,6,9trioxaundecan-1-amine (1 equiv., 2.52 g, 11.55 mmol) was added to the reaction and left under Argon gas flow at 55° C. overnight. The crude reaction was then purified by column chromatography with dichloromethane:ultra (22% MeOH in DCM with 3% NH40H) mixture 0% to 40% ultra on a 120 g ISCO silica column. The compound was characterized by mass spectroscopy and NMR to confirm the mass and purity.

Z2-Y12 analog synthesis. A suspension of sodium hydride (27.0 g, 675 mmol, 60% purity) in THF (200 mL) was cooled with an ice bath. Tetrahydro-4-pyranol (34.4 g, 337 mmol) was added in a dropwise fashion and stirred for 30 minutes at 0° C. 3-Bromoprop-1-yne (41.2 mL, 371 mmol, 80% purity) was then added in a dropwise fashion. The mixture was stirred overnight while allowed to warm to room temperature. The mixture was filtered over Cellite, washed with THF, and concentrated with Cellite under reduced pressure. The crude product was purified over silica gel (220 g) and eluted with Hexanes/EtOAc. The concentration of EtOAc in the mobile phase was increased from 0 to 25% to afford a yellow oil. The Z2-Y12 Analog was synthesized following the same procedure that was used for Z2-Y12 using the 4-(prop-2-yn-1-yloxy)tetrahydro-2H-pyran as reactant. The compound was characterized by mass spectroscopy and NMR to confirm the mass and purity.

Synthesis of methacryloyl Z2-Y12. Z2-Y12 (1.46 g, 5.06 mmol) was added to a 50 mL round bottom flask and purged with argon 3 times (10 mins each). 30 mL of anhydrous dichloromethane was added followed by triethylamine (769 mg, 7.60 mmol) and methacryloyl chloride (794 mg, 7.60 mmol) in a dropwise manner under argon atmosphere. The reaction was stirred for 5-6 hours followed by purification on a 40 g ISCO silica column using Hexanes/ethyl acetate (1:0 to 0:1) to obtain ˜1 gram of a colorless solid (Met-Z2-Y12). The compound was characterized by mass spectroscopy and NMR to confirm the mass and purity.

Synthesis of methacryloyl Z2-Y12 Analog. Z2-Y12 Analog (1.46 g, 5.06 mmol) was added to triethylamine (769 mg, 7.60 mmol) and methacryloyl chloride (794 mg, 7.60 mmol) in anhydrous dichloromethane (30 mL) under continuous nitrogen gas flow at room temperature, and the reaction was continuously stirred for for 5-6 hours. The crude reaction was then purified by liquid chromatography with a Hexanes/ethyl acetate (1:0 to 0:1) on a 40 g ISCO silica column. The compound was characterized by mass spectroscopy and NMR to confirm the mass and purity.

Synthesis of methacryloyl Z1-Y15. Z1-Y15 (1.9 g, 4.86 mmol) was added to methacryloyl chloride (0.794 g, 7.59 mmol) and 1.5 eq of triethylamine (0.754 g, 7.45 mmol) in anhydrous dichloromethane (30 ml) at room temperature. The reaction mixture was purged with argon for 30 minutes and continued for 5-6 hours before purification. The crude reaction was then purified by liquid chromatography with a dichloromethane: methanol mixture 5% to 10% on a 40 g ISCO silica column. The compound was characterized by mass spectroscopy and NMR to confirm the mass and purity.

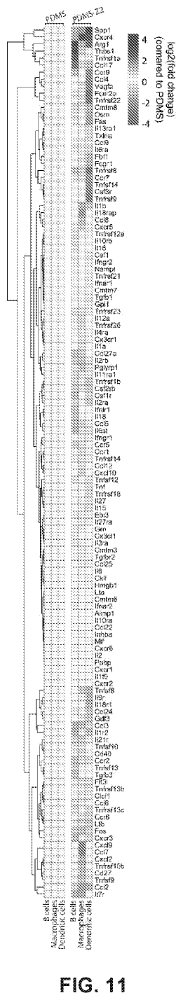

PDMS disk/pillar preparation & functionalization. PDMS implants were made with SYLGARD™ 184 silicone elastomer by mixing the base and curing agent in a ratio of 10:1 and letting sit 24 hours at room temperature in either sheets or pillar molds. The PDMS sheets were punched with 4 mm biopsy punches to make disks, or the pillar were removed from their molds, and plasma cleaned under general atmospheric air on each side for 1 minute at high Rf, 700-800 torr. Samples were then functionalized by one of two methods: Method 1) for the methacryloyl modified Z2-Y12 and Z2-Y12 Analog, the PDMS disks were incubating in a solution of DMSO:ACN (Sigma) (60:40) overnight under nitrogen gas flow. For the methacryloyl modified Z1-Y15 (0.2M), methacryloyl Z1-Y15 was added to DMSO (0.3 w/v %) and heated to 80° C. Separately Milli-Q H₂O was heated to 80° C. and added to the reaction dropwise for a final mixture of 1:4 DMSO:Milli-Q H₂O. The mixture was left stirring under reduced heat 40-50° C. under high stirring conditions (12000-1500 rpm). PDMS sheets were added to this mixture and allowed to stir overnight under Nitrogen gas. Disks were submerged in 1×PBS and left to soak for 10 minutes. Samples were then washed 3× with 1×PBS after overnight functionalization. This method was used for the experiments in FIGS. 9a-d and 10a-j. Method 2) PDMS disks were incubated in 0.2 M solutions of the small molecules in toluene with 5% DMSO for an hour followed by washing them three times in methanol, three times in ethanol, three times with sterile grade water, and finally again with sterile grade ethanol. This method was used for the experiments in FIGS. 11a-13c. Disks were then allowed to dry before cryo-storage. Functionalized disks were maintained at −80° C. until they were ready for implantation.

PTFE disk preparation and functionalization. PTFE septa (Supelco) were first oxidized following published protocol (Gabriel and Niederer, 2016). Z1-Y15 was then functionalized onto the PTFE-OH by first treating the disks with 50% (v/v) hexamethylene diisocyanate (HMDI) in dry THF for 2 hours. The disks were then washed with THF 3 times and dried using argon purging and slight heat (in hot oven for 5 mins at 50-55° C.). The disks were then hydrolyzed in water for 2-3 hr and dried. The disks were then treated with a solution of 20% (v/v) diethyleneglycol diglycidyl ether (diepoxide) [50 mM carbonate buffer, pH 9. 2] for two hours followed by a wash with carbonate buffers and mili-Q water. A solution of 0.1 M of Z1-Y15 in methanol was added to the modified disks overnight to attach the small molecule to the PTFE. The disks were finally washed with MeOH, EtOH, Mili Q and EtOH consecutively for three times each followed by drying with argon purging.

Animal studies. C57BL/6 and ApoE knockout mice (Jackson Laboratory or Charles River Laboratories) were used for in vivo studies. All animal experiments were approved by Rice University's (AAALAC 001676) or Loma Linda Institution Animal Care and Use Committee (IACUC). Animal work at Rice University was performed under IACUC protocol 20-200. Animal work at Loma Linda was performed under protocol. C57BL6 and ApoE mice (males ordered at 4-8 weeks of age) were allowed to acclimate for 1 week before any surgical manipulation.

Intraperitoneal space (IP) and subcutaneous mouse (SubQ) implants. After one week they were anesthetized with isoflurane at 3% isoflurane, 1.5 L/min O₂ in an induction chamber and then transferred to a nose cone. Animals were then ear punched for identification, given ophthalmic ointment, 0.1 mg/kg buprenorphine SR at 0.5 mg/mL, and their fur was trimmed from their abdomen (IP space implants) or back (SubQ implants). They were then scrubbed with IPA followed by iodine on their trimmed abdomens. This was repeated 3×. Animal surgeries were conducted with a No. 15T surgical blade (Sklar) and using sterile surgical gloves (CTI International).

For IP implants, a 1 cm incision was made low in the abdomen ending just above the pubic region, followed by a like-size incision in the IP wall. A gonadal fat pad was gently pulled out from the IP space using tissue forceps with teeth and placed on the exposed muscle wall. For 28 day scheduled implants disks were placed directly onto the gonadal fat pad with extreme care taken to not manipulate the face of the disks but rather the sides. A PDS II (Polydioxanone) suture was used to throw one suture at the disk edge to maintain the disk in place on top of the fat pad for 28 days. The gonadal fat pad with the implanted disk was then gently reinserted into the IP space so the disk was lying flat with one side facing the IP wall and the other facing the gonadal fat pad. This procedure was repeated for the gonadal fat pad on the mirrored side of the animal (2 gonadal fat pads per animal and 1 implant per fat pad site). For 1-day implants, the disk was gently placed on top of the gonadal fat pad by lifting the IP wall and inserting the disk into the exposed space. Extreme care was taken to not manipulate the face of the disks but always handle the sides and to ensure one face of the disk was flush with the IP wall and the other lay against the fat pad. One disk was implanted per gonadal fat pad, two gonadal fat pads per animal. For SubQ implants, an incision was made on the back of the mouse through the skin, and forceps were used to create two pockets in the subcutaneous space, one on each side of the mouse. A PTFE disk was placed into each pocket, one being unmodified and the other modified with Z1-Y15.

The muscle layer (for IP space implants), followed by the skin, was then sutured using a simple interrupted suturing method with PDS II sutures (Ethicon, Z503G). Lidocaine/Prilocaine ointment (2.5%) was then administered topically, and animals were placed in recovery cages to recover on a water heat pad set to 40° C. Animals were monitored 24 hours later for signs of pain and distress. For 28 day scheduled implants, the animals were monitored once daily for four consecutive days for signs of pain and distress, weight gain/loss, and wound incision status. For 1 day scheduled implants, animals were euthanized 24 hours after implantation surgeries.

Euthanasia and disk processing. Animals scheduled for euthanasia were first anesthetized using 1.5 L/min O₂ and 5% isoflurane. After animals were anesthetized, O₂ and isoflurane were stopped, and the gas line switched to CO₂ at 2 L/min. The CO₂ was left on until animals stopped showing signs of breathing (˜5 min, 25 g mouse). The animals were then removed from the induction chamber, cervically dislocated to ensure euthanasia, and underwent a dissection for disk retrieval. The disks for 1-day implants were located where placed, or within the relative location, and were pulled out using extreme care to not manipulate the face of the disks but always handle the sides. Samples were moved to cryotubes and frozen at −80° C. for later ToF-SIMS analyses. For 28-day explants, animals were euthanized in the same way. Entire fat pads were removed and placed in 35 mm dishes and maintained on ice. Samples were then gently manipulated to remove the single suture connecting the disk to the fat pad. Disks that were adhered to fibrotic tissue could not be removed from the fat pads they were fused to. Disks that had little/minimal or no fibrosis once the suture was removed freely came out of the fat pads. Day 28 explanted samples were then imaged using stereomicroscopy.

Mouse Brain Implantation. C57BL/6J mice were allowed to acclimate for greater than one week prior to surgical manipulation. After the acclimation period, 24 hours prior to surgery, the mice were premedicated with 5 mg/kg meloxicam SC for analgesia. 30 minutes prior to surgery, the mice were given 30 mg/kg trimethoprim-sulfamethoxazole PO for antimicrobial prophylaxis. The mice were then anesthetized with 3% isoflurane with 1 L/min O₂ in an induction chamber and then transferred to a nose cone with 1-3% inhaled isoflurane with 0.5 L/min O₂ for maintenance anesthesia. Depth of anesthesia was monitored testing for no response to toe pinch throughout surgery. Immediately following induction, the mouse was placed on a warming pad and maintained on the warming pad throughout surgery. The fur on the mouse head was trimmed with an electric razor and topical anesthetic with 2.5% lidocaine/prilocaine cream was applied for several minutes. The mice were then given 5 mg/kg meloxicam SC and 0.1 mg/kg buprenorphine SC for analgesia. The trimmed mouse scalp was then scrubbed with 7.5% povidone-iodine solution. Sterile neoprene surgical gloves (Medichoice) and sterile autoclaved instruments were used to maintain sterile technique throughout surgery. A No. 15 surgical blade (Cynamed) was used to make a 5 mm linear incision centered over the intended burr hole location 2.15 mm anterior to Bregma and 0.5 mm right of midline. Pericranium was cleared from the scalp and the mouse scalp was gently retracted with toothed forceps to allow visualization of Bregma. Following confirmation of Bregma and identification of the burr hole location, a high-speed drill with a 2 mm round drill bit was used to fashion a 2.5 mm wide burr hole. A No. 11 surgical blade (Cynamed) was used to make a 2 mm durotomy. Via this durotomy, the 2 mm custom implantable device was inserted orthogonally into the brain using toothless forceps. Extra care was taken to manipulate the flared implant head only and not disrupt the functionalized implant shank. Depending on the treatment group, each mouse received an non-functionalized PDMS (n=5) or Z1-functionalized (n=5) implant. The control group (n=5) underwent burr hole and durotomy only with no implantation. Sterile saline flushes were used to irrigate the field during surgery. The mouse scalp was carefully reapproximated over the implanted device and closed in single layer with interrupted 4.0 nylon suture (Matrix Wizard). Following surgery, the isoflurane was stopped, and the mice were placed in a warmed recovery chamber for approximately 30 minutes for close postoperative monitoring while waking from anesthesia and recovering from surgery. Following recovery, the mice were housed individually. Mice were checked every 2 hours for the first 4 hours after surgery, then every 12 hours per the animal care facility guidelines. Postoperatively, mice were dosed with 5 mg/kg meloxicam SC for 72 hours and 0.1 mg/kg buprenorphine every 8-12 hours for 48 hours for pain control. No mice required additional dosing due to concerns with uncontrolled pain.

Euthanasia and Mouse Brain Implant Processing. Two weeks after surgical implantation, animals underwent euthanasia. For the euthanasia procedure, the mice were anesthetized with 3% isoflurane with 1 L/min O₂ in an induction chamber and then transferred to a nose cone with 1-3% inhaled isoflurane with 0.5 L/min O₂ for maintenance anesthesia. Since the animals were sacrificed following surgery, no additional analgesic premedication or antibiotics were necessary. Following induction, the prior incision was reopened, and the implanted device was carefully removed and handed off to another member of the research team for post-explanation processing while the first team member completed the mouse work. Once receiving the explanted device, the second team member immediately dabbed the explanted device gently on a Kimwipe (Kimtech) to wick off excess fluid, placed it directly into liquid nitrogen for flash freezing, and then transferred it into a collection tube that was stored in liquid nitrogen while completing the remaining mouse surgeries. Following implanted device removal, the mouse was decapitated using a rodent guillotine. The entire head was then briefly washed in ice cold DPBS with calcium and magnesium for 3 washes and then transferred into a collection tube with 4% paraformaldehyde. The collection tubes containing the mouse heads were stored in an ice bath while completing the remaining mouse surgeries. Once all surgeries were completed, the collection tubes containing the mouse heads in 4% paraformaldehyde were transported to the 4° C. fridge for 48 hours for fixation, and the collection tubes containing the explanted devices were transported to the −80° C. freezer for storage until they were shipped on dry ice to Rice University for surface lipid analysis.

Mouse Brain Cryoprotection & Cryostat Sectioning. After 48 hours of fixation in 4% paraformaldehyde, the mouse brains were washed in ice cold 1×PBS for 3 washes (5 minutes per wash) to clear fixative. Using rongeurs, forceps, and scissors, the mouse brain was carefully dissected from the skull, and the dissected brain was washed again in 1×PBS for 3 washes (5 minutes per wash) to ensure all fixative cleared. The mouse brains were placed in 15% sucrose solution until sinking and then transferred to 30% sucrose solution until sinking. After sinking in 30% sucrose, the brains were dabbed gently on a Kimwipe (Kimtech) to remove excess fluid, and then transferred into a peel away embedding mold (Fischer Scientific) lined with OCT embedding medium (TissueTek). Additional OCT was applied to cover the entire brain with OCT. Once covered in OCT, the embedding mold containing the brain and OCT was frozen over dry ice, wrapped tightly in aluminum foil, and stored the −80° C. freezer. Of note, the implant insertion site was marked on the embedding mold for reference during tissue sectioning. Using a cryostat (Leica) set at −20° C., each brain was coronally sectioned into 10 m thick sections and mounted onto microscope slides (Fisherbrand). The slides with the mounted brain sections were stored in the −20° C. freezer.

Immunohistochemistry, H&E Staining, & Microscopy. For immunofluorescence staining, the slides with the mounted brain sections were washed with 1×PBS for 3 washes (5 minutes per wash) and then blocked in 5% donkey blocking serum (Abcam ab7475) with 0.3% Triton-X-100 (Milipore) and incubated for 2 hours at room temperature. After further washing steps using 1×PBS with 0.5% Tween 20 (PBST; Thermo Scientific) for 3 washes (5 minutes per wash), the mounted slides were incubated with the primary antibodies in 1×PBST overnight in the 4° C. fridge. The primary antibodies used were GFAP (1:250; Novus Biologicals NB100-53809) and CD68 (1:250; Novus Biologicals NBP2-33337). The following day, the mounted slides were washed with 1×PBST for 3 washes (5 minutes per wash), and then incubated with the secondary antibodies in 1×PBST for 2 hours at room temperature. The secondary antibodies used were AlexaFluor 568 (1:1000; Fischer Scientific A-11057) and AlexaFluor 647 (1:1000; Fischer Scientific A48272). After further washing steps in 1×PBST for 3 washes (5 minutes per wash), the mounted slides were incubated with DAPI (1:1000; Abcam ab228549) in 1×PBST at room temperature for 5 minutes. After final washing steps with 1× PBST for 3 washes (5 minutes per wash), ProLong Gold antifade mountant (Fischer Scientific) was applied and the slides were coverslipped (Fisherbrand). The coverslipped slides were stored in 4° C. fridge.

For H&E staining, the slides with the mounted brain sections were dipped 5 times in 70% ethanol, 7 times in water, and then submerged in hematoxylin (Leica) once for 30 seconds. The mounted slides were then washed in running tap water for 30 seconds, dipped 7 times in 1% ammonium hydroxide solution in water, and washed again in running tap water for 30 seconds. The mounted slides were then dipped twice in 1% hydrogen chloride in 70% ethanol, and then washed again in running tap water for 30 seconds. The mounted slides were then dipped 7 times in 95% ethanol, 3 times in eosin (Leica), 7 times in 70% ethanol, 7 times in 95% ethanol, 7 times in 100% ethanol twice (using a separate container of ethanol for each set of dips), and 7 times in xylene twice (using a separate container of xylene for each set of dips). Xylene-compatible acrytol mounting medium (Leica) was applied and the slides were then coverslipped (Fisherbrand). The coverslipped slides were stored at room temperature.

Imaging was completed using the Olympus OlyVIA Virtual Microscope (Olympus). Images were captured on the microscope and imported into Photoshop (Version 22, Adobe). The GFAP images were imported into the green filter, the CD68 images were imported into the red filter, and the DAPI images was imported into the blue filter, and the images were merged via overlay for analysis.

Human IP catheter explants. De-identified clinical specimens of catheter explants collected from recipients of peritoneal shunt implants following institutional review board approval at Loma Linda University and acquisition of informed consent. Catheters were removed upon failure of the device. Catheters were dabbed against sterile gauze immediately following removal from the body to wick off CSF/peritoneal fluid while minimizing handling of the explanted device. Catheters were then dropped directly into liquid nitrogen. Once flash-frozen in liquid nitrogen, the flexible catheters straightened and stiffened, allowing them to be easily placed in collection tubes. Collection tubes were then returned to liquid nitrogen until the conclusion of the surgery. Catheters in collection tubes were transported to −80° C. freezer from the operation room after surgery and shipped on dry ice to Rice University, Houston Texas.

ToF-SIMS analysis. After explant at 24 hours, the PDMS disks were flash-frozen in liquid nitrogen and stored at −80° C. until time for analysis. A top mount holder (from IONTOF GmbH) was used to handle samples due to the softness and the possible fragility of the specimens. Samples were held in place using double-face carbon tape that was fixed to the holder. Then the sample holder has been attached to the transfer arm using a bayonet fitting and introduced in the load lock chamber until the vacuum pumping reaches a vacuum of 5.10⁻⁶ mbar ensuring an appropriate detection limit (typically over a few ppm).

ToF-SIMS instrument parameters & instrument software. Negative high resolution mass spectra imaging was performed using a TOF-SIMS NCS instrument, which combines a TOF.SIMS 5 instrument (ION-TOF GmbH, Münster, Germany) and an in-situ Scanning Probe Microscope (NanoScan, Switzerland) at the Shared Equipment Authority from Rice University.

Spectrometry. A bunched 30 keV Bi₃⁺ ions (with a measured current of 0.2 pA) was used as the primary probe for analysis (scanned area 90×90 μm²) with a raster of 128×128 pixels. A charge compensation with an electron flood gun was applied during the analysis. Low energy electron beams were flooded onto the sample, which was permitted to compensate the charge buildup on the surface. An adjustment of the charge effects was also done using a surface potential of −10 V. The cycle time was fixed to 200 μs (corresponding to m/z=0-3649 a.m.u mass range).

2D imaging. A bunched 60 keV Bi₃⁺⁺ ions (with a measured current of 0.3 pA) using the “fast image mode” configuration with a 100 ns pulse width for the imaging was utilized. Analysis cycles with Bi₃⁺⁺ ions were used to acquire images (2048×2048 pixels), scanning an area of 90×90 μm². The images have been binned to enhance the signal-to-noise ratio. All data were analyzed using the IONTOF Surface Lab 7.1 software. After a mass calibration of the mass spectra using C⁻, CH⁻, O⁻, OH⁻, F⁻, Si, SiH⁻, SiH₂⁻, and SiO⁻, the “compound search” tool was used to select and identify the characteristic peaks of lipid residues and small molecules functionalized on the materials.

ToF-SIMS computational analysis methods. Peaks were extracted from the ToF-SIMS using the ION-TOF software and normalized for comparison by normalizing ion counts to the total ion intensity. Peak lists generated by the ION-TOF software were combed through and lipids identified using the lipid fingerprint spectra available through the ION-TOF library of mass spectra. A heatmap was then generated by comparing the lipid-identified peaks using MATLAB.

Statistics. Histograms, box-and-whisker plots, and corresponding statistics were generated using Prism 7 or MATLAB. One way ANOVA tests, when conducted, were done with Tukey's or Fisher's method for multiple comparisons.

scRNA-seq analysis. PDMS and PDMS-Z2-Y12 disks were implanted into the IP space of the mice at the gonadal fat pads. After 2 weeks, implants and washed with sterile PBS to isolate cells form the surface. Resulting cells were sorted on a Sony MA900 Cell Sorter to filter out dead cells, and libraries were generated from the remaining cells on the 10× Chromium platform for 5′ scRNA-seq according to manufactures' protocol. The libraries were then sequenced on a Illumina NovaSeq 6000 flow cells and processed on the 10× Genomics Cloud Analysis platform with the Cell Ranger v6.1.2 pipeline. The R package Seurat V4 (Satija Lab) (Hao et al., 2021) was used to cluster the transcripts by K-means clustering and generate UMAP coordinates which were visualized on the Loupe Browser 6.0. Clusters were identified as immune cell populations by expression of common immune cell markers, Cd79a (B cells), Fn1 (macrophages), Cd3d (T/NK cells), dendritic cells (Cd209a), and neutrophils (S100a9). Volcano plots and heatmap generated with Seurat V4.

Lipid spin-coating of PDMS disks. Lipids were dissolved in mixtures in 100% ethanol at an end concentration of 20 mg/ml for total lipid in solution. One lipid solution contained equal amounts of sphingomyelin, phosphatidylinositol, phosphatidylethanolamine, and phosphatidylcholine (lipids that deposit on immune-evasive materials), while the other solution contained equal amounts of palmitoleic acid, linolenic acid, linoleic acid, and oleic acid (lipids similar to those that deposit of immunogenic material). PDMS and PDMS-Z2-Y12 disks were cleaned by nitrogen gas and then these lipid solutions were spin-coated on the disks via spin coating by coating pipetting the lipid solution onto the PDMS disks and then following a two-step spin process: A first, slower rotation allows the solution to cover the surface homogeneously (1 second at 500 rpm, acceleration at 1000 rpm/second), followed by a second step that enforces quick-drying and tossing of the spare solution (30 seconds at 3000 rpm, acceleration at 2500 rpm/second). To remove all traces of ethanol, the samples were exposed to high vacuum over 24 h. Lipid coating was validated via ToF-SIMS analysis. The samples were kept refrigerated until needed.

Macrophage harvest and isolation. Mice were euthanized via CO₂ euthanasia under isoflurane anesthesia. The abdomen of each mouse was soaked with 70% alcohol, and then a small incision was made along the midline with sterile scissors. The abdominal skin was retracted manually to expose the intact peritoneal wall. A slight incision was made in the center of the peritoneal wall and 1 mL of ice-cold harvest medium (PBS) was injected into the IP space with a pipette. The incision was held closed, and the mouse was shaken. Using the same pipette, fluid was aspirated from the peritoneum. The pipette was moved away from the viscera to cause tenting of the peritoneal wall, and fluid was withdrawn slowly. This process was repeated until 10 mL of harvest medium was used. The harvested peritoneal fluid was dispensed into a 50-ml conical polypropylene centrifuge tube on ice.

The harvested fluid was centrifuged in a refrigerated centrifuge 10 min at 500 g, 4° C. to isolate the peritoneal exudate cells (PEC). The supernatant was discarded, and then the cells were treated with RBC lysis buffer (Sigma). DMEM/F12-10 media was added to stop the lysis budder, and the cells were then spun down for 5 mins at 500 g, 4° C. The supernatant was discarded, and cells were resuspended in cold DMEM/F12-10 media by gently tapping the bottom of the tube and pipetting up and down. Cells were counted using a 1:1 ratio of trypan blue to cell stock. Cell concentration was adjusted to 3×10⁶ total peritoneal cells/ml and keep on ice. From here cells were cultured in 35 mm cell culture dishes that had one of the following on the bottom: PDMS, PDMS coated with the immunogenic lipids, PDMS-Z2-Y12, PDMS-Z2-Y12 coated with the immune-evasive lipids. To obtain monolayers of peritoneal macrophages, the PECs were allowed to adhere to the substrate by culturing them for 2 hours at 37° C. Nonadherent cells are removed by gently washing three times with warm PBS. At this time, cells still attached to the substrate should be greater than 90% macrophages. The presence of macrophages was confirmed via ELISA of TNF-α expression and immunostaining with CD68 and F4/80 stains (not shown). Macrophages were cultured for 24 hours on the substrates before either being fixed or lifted and having the RNA extracted from them.

Vinculin staining and confocal imaging. Macrophages cultured on PDMS were fixed by incubating in 4% paraformaldehyde for 10 minutes. Cells were then permeabilized with a 10-minute incubation with 0.1% Triton X-100 and then blocked with a 1% BSA solution for 30 minutes. Cells were then incubated with anti-vinculin antibody (Sigma-Aldrich, SKU: V9131, diluted 1:200) for 1 hour, followed by a 1-hour incubation with secondary antibody labeled with Atto 594 (Sigma-Aldrich, SKU: 76085, diluted 1:200). Cells were stained with DAPI NucBlue™ Fixed Cell ReadyProbes™ Reagent (ThermoFisher, catalog number: R37606) following the manufacturer's protocol.

Confocal images of macrophages on functionalized and non-functionalized disks were acquired with a Nikon A1 inverted confocal microscope (Nikon Instruments Inc., Melville, NY) using 60×/1.27 WI Plan Apo IR (OFN25 DIC N2) objective. Optical setup to determine fluorescence of DAPI-labeled macrophages: 400 nm laser line (100% relative power), 405 nm main dichroic mirror, emission filter long-pass 495 nm (450/50). Optical setup to determine fluorescence of vinculin antibody-labeled macrophages: 486 nm laser line (40% relative power), 488 nm main dichroic mirror, emission filter long-pass 560 nm (525/50).

Bulk ex-vivo RNA sequencing. 1M mouse macrophages per sample were extracted from C57BL/6 mice and cultured on PDMS disks containing lipid and small molecule components as described above. After 24 hours, RNA was isolated with the Qiagen RNeasy Plus Mini Kit (Cat No/ID: 74106) according to the manufacturer's protocol. Ribosomal RNA depletion, library preparation, and sequencing were conducted at GENEWIZ LLC (South Plainfield, NF, USA). RNA quantification was performed by Qubit 2.0 fluorometer (Life Technologies) and RNA integrity was measured by Agilent Tapestation 4200 (Agilent Technologies). Sequencing libraries were prepared using the Illumina TruSeq Stranded Total RNA with Illumina Ribo-Zero Plus rRNA Depletion Kit (Illumina) according to the manufacturer's protocol. Sequencing libraries were clustered on three lanes of a flow cell and ran on the Illumina HiSeq with a 2×150 bp paired-end configuration.

Bulk ex-vivo RNA-seq data analysis. Quality control of raw reads was performed using FastQC (version 0.11.9) and aggregated using MultiQC (version 1.9). Adapter reads were removed using Trimmomatic (version 0.39) (Bolger et al., 2014). Reads were pseudo-aligned to the GRCm38/mm10 genome using Kallisto (version 0.44.0) with an index type containing both mRNA and ncRNAs (Bray et al., 2016). Abundances were quantified using the processed read files and 100 bootstraps with default parameters. Differential gene expression levels between groups were identified using DESeq2 calling significance with an adjusted p-value for multiple hypothesis testing (p_{adj})<0.05 cutoff (Pimentel et al., 2017; Soneson et al., 2015; Love et al., 2014). Differential transcript expression levels between groups were identified using Sleuth calling significance with an adjusted p-value (p_{adj})<0.05 cutoff.

Z2-Y12, NMR and Mass Spec Data. ¹H (600 MHz; DMSO-D6): 1.59 (4H, m, tetrahydropyran), 1.77 (2H, m, tetrahydropyran), 3.68 (1H, m, tetrahydropyran), 3.73 (1H, m, tetrahydropyran), 4.32 (2H, m, methylene), 4.57 (1H, s, tetrahydropyran), 4.65 (2H, s, methylene), 7.28 (2H, s, benzene), 7.62 (2H, s, benzene), 8.08 (1H, s, triazole), 8.49 (2H, s, amine).

¹³C (600 MHz; DMSO-D6): 19.4 (CH3, tetrahydropyran), 25.4 (CH2, tetrahydropyran), 30.5 (CH2, tetrahydropyran), 44.6 (CH2, aliphatic), 62.6 (CH2, aliphatic), 120.7 (CH, triazole), 130.0 (CH, benzene), 136.2 (CH, benzene).

ESI: m/z 289 [M+H], m/z 577 [2M+H].

Z1-Y15, NMR and Mass Spec Data. ¹H (600 MHz; DMSO-D6): 2.90 (4H, m, methylene), 3.07 (2H, m, methylene), 3.53 (12H, m, methylene), 3.61 (2H, s, methylene), 3.67 (2H, m, methylene), 3.83 (2H, m, methylene), 3.90 (2H, m, methylene), 7.55 (1H, s, triazole).

¹³C (600 MHz; DMSO-D6): 41.5 (CH2, aliphatic), 50.3 (CH2, cyclohexane like), 52.1 (CH2, aliphatic), 53.5 (CH2, cyclohexane like), 69.4 (CH2, aliphatic), 70.2 (CH2, aliphatic), 70.5 (CH2, aliphatic), 124.0 (CH, triazole).

ESI: m/z 392 [M+H].

Z2-Y12A, NMR and Mass Spec Data. ¹H (600 MHz; DMSO-D6): 1.58 (2H, m, tetrahydropyran), 1.83 (2H, m, tetrahydropyran), 3.40 (1H, m, tetrahydropyran), 3.64 (2H, m, tetrahydropyran), 3.75 (2H, m, tetrahydropyran), 4.02 (2H, s, methylene), 4.30 (2H, s, methylene), 7.33 (2H, s, benzene), 7.61 (2H, s, benzene).

¹³C (600 MHz; DMSO-D6): 32.0 (CH2, tetrahydropyran), 47.2 (CH2, aliphatic), 60.2 (CH2, tetrahydropyran), 65.2 (CH2, aliphatic), 73.6 (CH, tetrahydropyran), 120.4 (CH, triazole), 129.2 (CH, benzene), 146.1 (C, triazole).

ESI: m/z 289 [M+H], m/z 577 [2M+H].

Met-Z2-Y12, NMR and Mass Spec Data. ¹H (600 MHz; DMSO-D6): 1.52 (4H, m, tetrahydropyran), 1.64 (2H, m, tetrahydropyran), 1.91 (3H, s, methyl), 3.52 (1H, m, tetrahydropyran), 3.82 (1H, m, tetrahydropyran), 4.39 (2H, s, methylene), 4.59 (1H, d, tetrahydropyran), 4.79 (2H, s, methylene), 5.40 (1H, s, ethylene), 5.75 (2H, s, ethylene), 7.46 (2H, s, benzene), 7.85 (2H, s, benzene), 8.61 (1H, s, triazole), 8.80 (1H, s, amide).

¹³C (600 MHz; DMSO-D6): 19.2 (CH3, aliphatic), 19.4 (CH3, tetrahydropyran), 25.4 (CH2, tetrahydropyran), 31.2 (CH2, tetrahydropyran), 42.3 (CH2, aliphatic), 61.7 (CH2, aliphatic), 120.5 (CH, triazole). ESI: m/z 357 [M+H], m/z 379 [M+Na], m/z 713 [2M+H].

Met-Z1-Y15, NMR and Mass Spec Data. ¹H (600 MHz; DMSO-D6): 2.09 (3H, s, methyl), 2.89 (4H, m, methylene), 3.09 (2H, m, methylene), 3.35 (12H, m, methylene), 3.44 (2H, s, methylene), 3.48 (2H, m, methylene), 3.58 (2H, m, methylene), 3.76 (2H, m, methylene), 5.77 (2H, d, ethylene), 8.01 (1H, s, triazole).

¹³C (600 MHz; DMSO-D6): 19.1 (CH3, methyl), 40.1 (CH2, aliphatic), 49.8 (CH2, cyclohexane like), 50.9 (CH2, aliphatic), 51.8 (CH2, cyclohexane like), 69.3 (CH2, aliphatic), 70.1 (CH2, aliphatic), 119.6 (CH2, ethylene), 124.8 (CH, triazole).

ESI: m/z 460 metRZA15 [M+H], m/z 482 [M+Na].

Met-Z2-Y12A, NMR and Mass Spec Data. ¹H (600 MHz; DMSO-D6): 1.61 (2H, m, tetrahydropyran), 1.76 (2H, m, tetrahydropyran), 1.92 (3H, s, methyl), 3.44 (1H, m, tetrahydropyran), 3.69 (2H, m, tetrahydropyran), 3.73 (2H, m, tetrahydropyran), 4.58 (2H, s, methylene), 5.40 (1H, s, ethylene), 5.75 (1H, s, ethylene), 7.46 (2H, s, benzene), 7.72 (2H, s, benzene).

¹³C (600 MHz; DMSO-D6): 18.7 (CH3, aliphatic), 32.4 (CH2, tetrahydropyran), 43.1 (CH2, aliphatic), 61.3 (CH2, tetrahydropyran), 65.7 (CH2, aliphatic), 77.0 (CH, tetrahydropyran), 120.6 (CH, triazole), 129.1 (CH, benzene), 146.5 (C, triazole).

ESI: m/z 357 [M+H], m/z 379 [M+Na].

### Example 2—Results

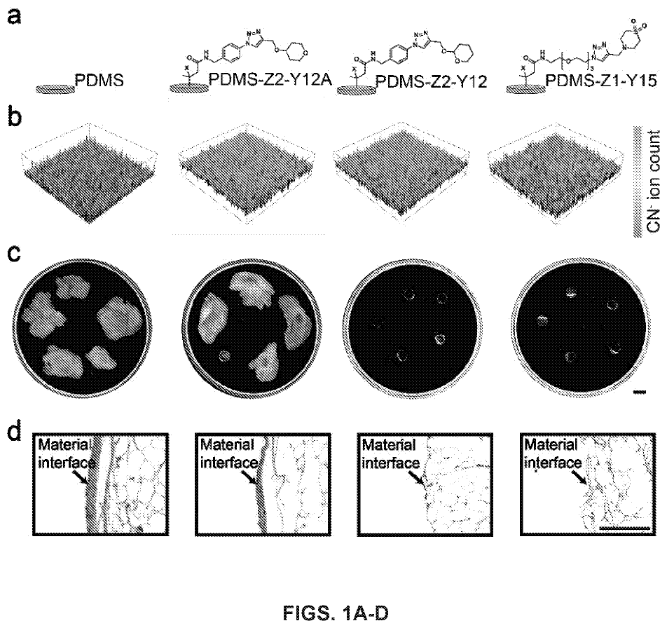

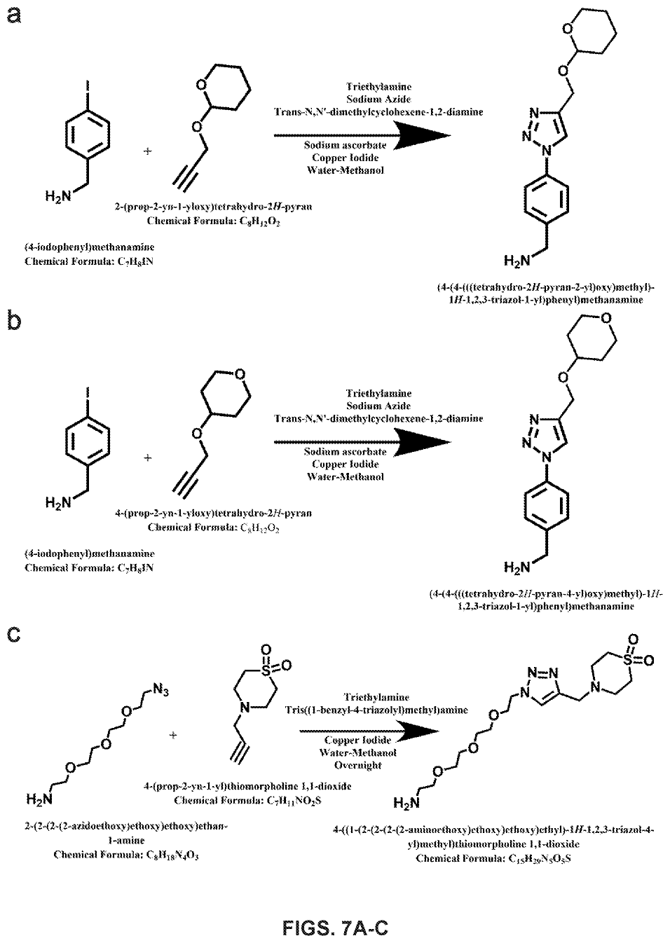

Surface molecular engineering using immunomodulatory triazoles conveys anti-FBR properties to PDMS implants. Surface-modified and unmodified implants were fabricated by functionalizing polydimethylsiloxane (PDMS), a common material in many medical devices including catheters (Yoo et al., 2018; Meacham et al., 2008; Mata et al., 2017), with the small molecules Z1 and Z2 (Vegas et al., 2016a). Surface functionalization with these molecules have previously been shown to mitigate FBR on alginate implants and other medical device implants (Vegas et al., 2016a; 2016b; Xie et al., 2018; Bose et al., 2020; Ghanta et al., Bochenek et al., 2018). Additionally, the inventors synthesized a non-functional structural analog of Z2, referred to as Z2A, was used as a control (FIG. 1a). Z2 and Z2A were synthesized by combining (4-iodophenyl)methanamine with either 2-(prop-2-yn-1-yloxy)tetrahydro-2H-pyran or 4-(prop-2-yn-1-yloxy)tetrahydro-2H-pyran, respectively in a click-chemistry reaction (FIGS. 7a-b). Z1 was synthesized by combining 2-(2-(2-(2-azidoethoxy)ethoxy)ethoxy)ethan-1-amine with 4-(prop-2-yn-1-yl)thiomorpholine 1,1-dioxide in a click-chemistry reaction (FIG. 7c). Syntheses were validated via mass spectrometry and NMR. Z2, Z2A, and Z1 were then functionalized to the surface of PDMS by adding a methacrylate group to the molecules and reacting them with plasma-treated surfaces of PDMS. To confirm the small molecule surface modification, the surfaces of all functionalized and non-functionalized PDMS disks were analyzed for the small molecules with ToF-SIMS. Carpet plot renderings of the CN⁻ ion intensity, an ion that is present in the fragmentation patterns all the small molecules, over a 300 μm×300 μm area showed that the functionalized PDMS disks were uniformly functionalized with the molecules (FIG. 1b). The amount of small molecule on each disk and the hydrophobicity of each disk was also found to be the same across conditions (FIG. 9).

To validate that Z1 and Z2 can both protect PDMS implants against fibrosis, and that Z2-Y12Analog does not, PDMS disks functionalized with these molecules were implanted into the gonadal fat pads of C57BL/6 adult male mice, a pro-fibrotic mouse model (Doloff et al., 2017), for 28 days. Adherent peripheral tissue could be found attached to the non-functionalized and PDMS-Z2A disks after 28 days, signifying biomaterial-induced FBR and fibrosis. In contrast, virtually no adherent peripheral tissue was found on chemically-modified PDMS disks functionalized with Z1 or Z2 (FIG. 1b), indicating the small molecules confer persistent protection against FBR and fibrosis. Furthermore, histology of tissues around the implants showed that the Z1 and Z2 functionalization prevented the development of an interfacial fibrotic layer on the implant surface, which was clearly visible in the PDMS and PDMS-Z2-Y12A controls (FIG. 1c).

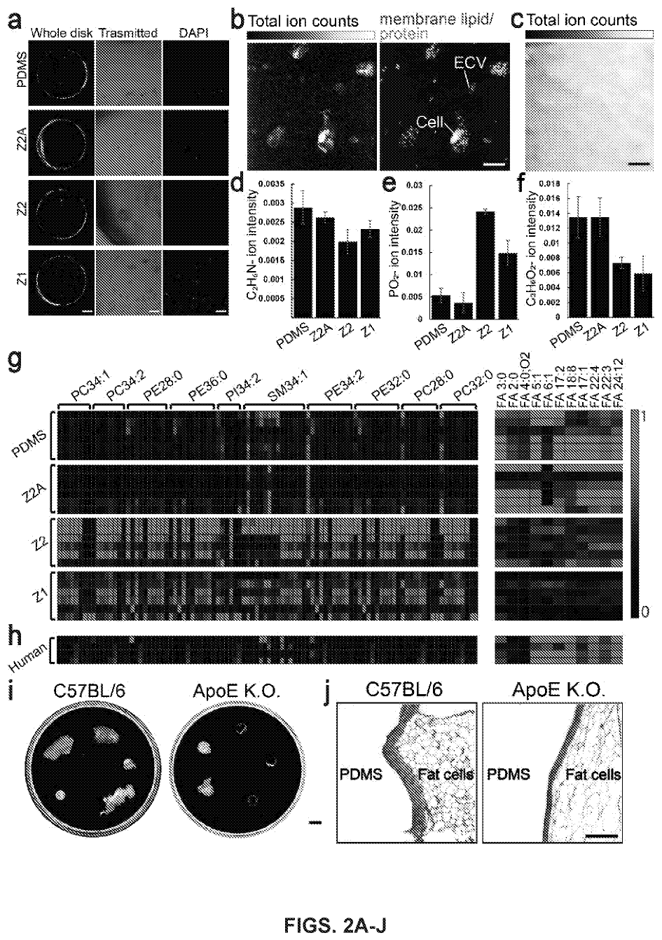

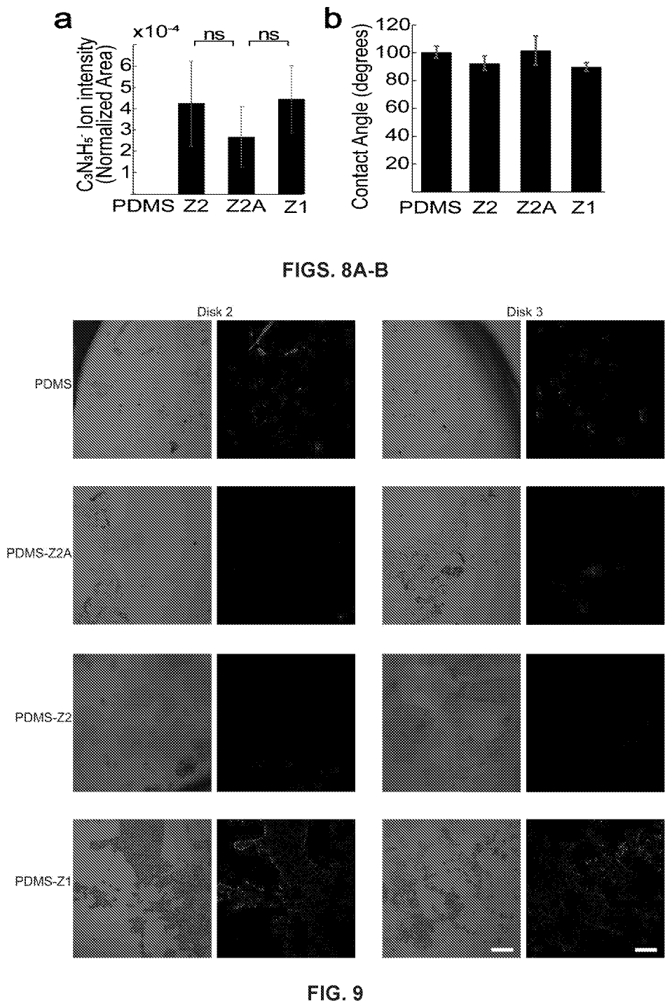

Phospholipids are selectively enriched on implants functionalized with anti-fibrotic small molecules. The inventors next analyzed how Z1 and Z2 impacted the composition of lipid molecules that associate with PDMS implants. For these experiments, functionalized and unfunctionalized PDMS disks were implanted into the gonadal fat pads of C57BL/6 mice for a 24-hour period. Adherent fibrotic tissue was not observed on any of the implants over this brief time, and only low numbers of sparsely distributed cells were observed on explanted disks (FIG. 2a and FIGS. 8a-b). ToF-SIMs analyses of sparsely distributed cells revealed that the cells were surrounded by patches of molecules that contained the same ion signatures as a cell membrane, suggesting that the cells were leaving parts of their membrane on the implant surfaces, presumably as extracellular vesicles (FIG. 2b). Consistent with optical microscopy, most of an implant's surface did not have cells, and displayed relatively uniform distribution of ion signals (FIG. 2c). To focus on the lipids that were on the surface of the disks, the rest of the analysis was limited to these acellular regions.

The total amount of phospholipids, fatty acids, and proteins that were deposited on the disk's during implantation was quantified examining the counts of ion fragments in ToF-SIMS spectra that can be attributed to these biomolecules. Total levels of proteins were similar across all chemically modified and control implants (FIG. 2d), suggesting that the immune-evasive properties conveyed by these antifibrotic modifications is not dependent on the level of protein deposition. Total levels of phospholipids (PLs) and fatty acids (FAs) were next determined by quantifying the intensities of the PO₂⁻ and C₃H₆O₂⁻ ions, which are common to PLs and FAs, respectively, and absent from the PDMS background. Comparison of these ion counts showed that PDMS functionalized with the anti-fibrotic molecules had ˜400% more PLs than the control conditions (FIG. 2e). In contrast, PDMS functionalized with the anti-fibrotic molecules had ˜50% less FAs than the control conditions (FIG. 2f). Taken together, these results suggest that these anti-fibrotic modifications convey immune-evasive properties to implants by modulating lipid deposition.

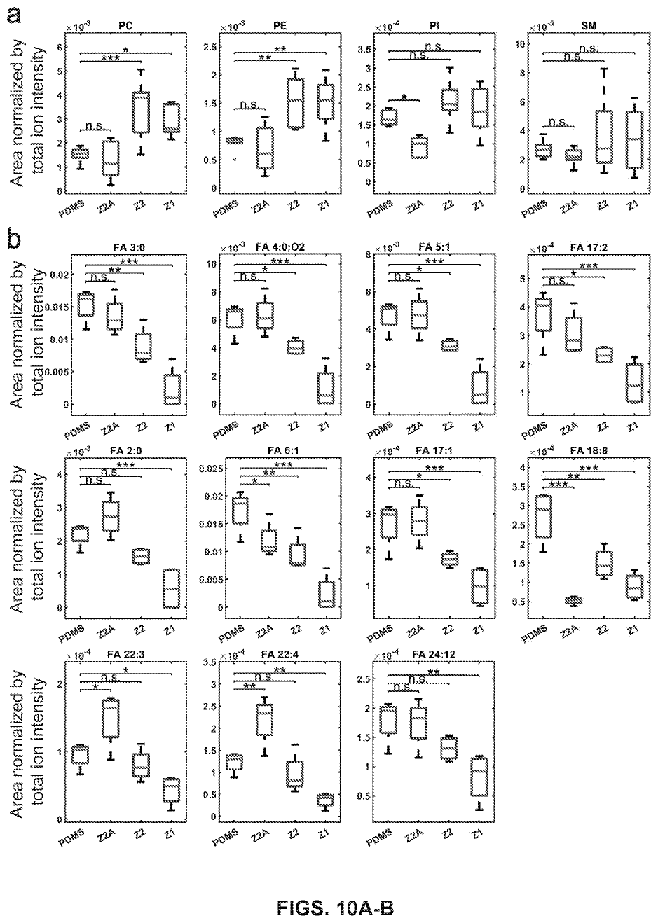

Further analyses of the composition of lipids that deposited on the implants, utilizing methods and reference spectra developed by Taylor et al. (2018) and Johansson et al. (2006), suggest the increase in PO²⁻ ions on the Z2 and Z1 implants stems from the deposition of phosphatidylcholine (PC), phosphatidylethanolamine (PE), phosphatidylinositol (PI), and sphingomyelin (SM) (FIG. 2g and Supplementary Table 1). The prevalence of these lipid is evident in column-scaled heatmaps that display the intensities of multiple ion fragments associated with these different phospholipid sub-species. Further quantitative comparisons of lipid enrichment levels were performed by comparing ion intensities of peaks representative of each phospholipid group (for PC, PE, PI and SM). These results were presented as box-and-whisker plots (FIG. 10a), and show that phosphatidylcholine, and phosphatidylethanolamine levels are significantly increased on the anti-fibrotic conditions compared to the fibrotic conditions (ANOVA, p≤0.05). PI and SM follow this same pattern but statistical differences were not found to be significant. A comparison of the means of each condition revealed that the anti-fibrotic conditions had a ˜2 fold enrichment of PC, and PE compared to the fibrotic conditions.

The distributions of lipids on fibrotic PDMS implants are enriched in fatty acid molecules. To identify the specific fatty acids that were preferentially depositing on the fibrotic materials, the inventors extended their analysis by cross referencing the ToF-SIMs spectra with the LIPID MAPS® structural database (LMSD) using methods developed by Passarelli et al. (2011). This analysis identified 11 FAs that were enriched on both the non-functionalized and PDMS Z2-Y12A functionalized controls. FA lipids notably do not have phosphate head groups, so their levels would therefore not be reflected by PO₂⁻ counts as is the case with phospholipids. Nevertheless, FA enrichment on the control implants can be seen in column-scaled heatmaps of ion intensities (FIG. 2g, and Supplementary Table 2), which display higher FA fragment counts for the controls compared to the Z2 and Z1 functionalized disks. Further, quantitative comparisons of the lipids show that ions from 5 of these fatty acids are enriched between 2 and 4-fold on the fibrotic controls (ANOVA, p≤0.05). The other fatty acids followed this same pattern, but the differences were not significant in this analysis (FIG. 2b).

To assess whether the relationship between fibrosis and FA deposition also translates to human patients, the inventors next performed ToF-SIMs analysis on explanted human intraperitoneal catheters that were also made of PDMS. These catheter devices were explanted from patients because they induced fibrosis and became non-functional. After removing the tissue layer from the device, 9 out of the 11 FA that were enriched on the mouse implants were detected by ToF-SIMS. Moreover, consistent with the inventors' data in mice, phospholipids ions were not detected (FIG. 2h), suggesting that this lipid response also occurs in humans.

Disrupting lipid metabolism attenuates biomaterial induced fibrosis. To further probe the role of lipids in biomaterial induced fibrosis in vivo, the inventors examined the fibrotic response induce by PDMS implants in Apolipoprotein E (ApoE) knockout C57BL/6 mice. ApoE binds lipids and forms a lipoprotein complex that assists lipid transport in the bloodstream. Lipid concentrations are increased in plasma in double ApoE knockout mice, which also display a systemic inflammatory response (Zhang et al., 1992; Sasso et al., 2016). Yet, despite this systemic inflammation, PDMS disks displayed a weaker biomaterial induced fibrosis after a 28-day implantation period. Only 2 out of 5 disks displayed adherent peripheral tissue in the ApoE knockout mice, in contrast to all of the disks in the C57BL/6 mice (FIGS. 2i-j). Thus, the altered lipid metabolisms and increased lipid circulation appears to result in a weakened biomaterial-induced fibrotic response.

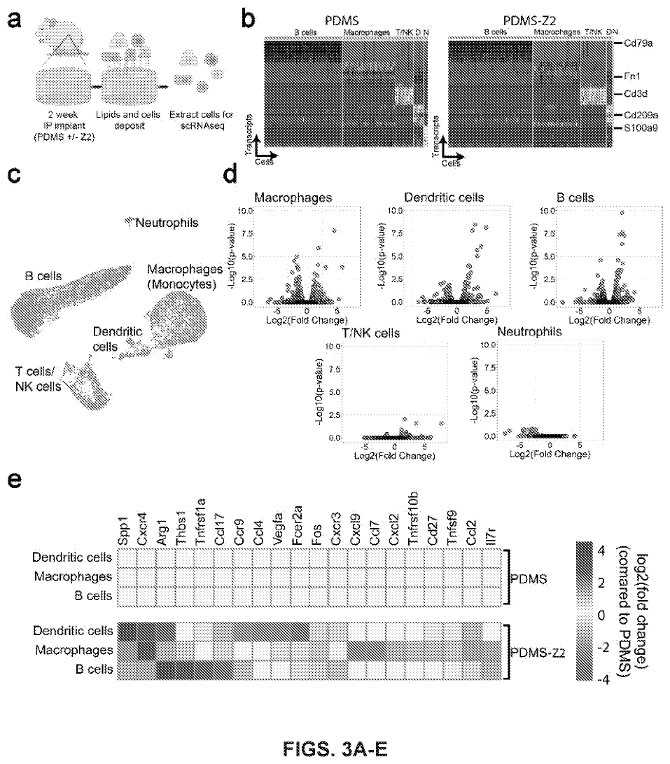

Immunological analysis of anti-fibrotic surface modifications. To investigate immune cell interactions with the chemical-modified, anti-fibrotic implants, cells were extracted from the space around PDMS and PDMS-Z2 2-week implants and submitted for single-cell RNA-sequencing (scRNA-seq) to examine the distribution and transcriptional activation of immune cells (FIG. 3a). Immune cell subtypes were identified via k-means clustering of single cell transcriptomes. This analyses revealed 5 major clusters corresponding to B cells, macrophages, dendritic cells, T/Natural Killer cells, and neutrophils, which could be visualized in uniform manifold approximation and projection (UMAP) plots (FIGS. 3b-c). Significant transcriptional differences were observed with B-cells, dendritic cells and macrophages depending on whether the surface was functionalized with Z2 or not (FIG. 3e, FIG. 11).

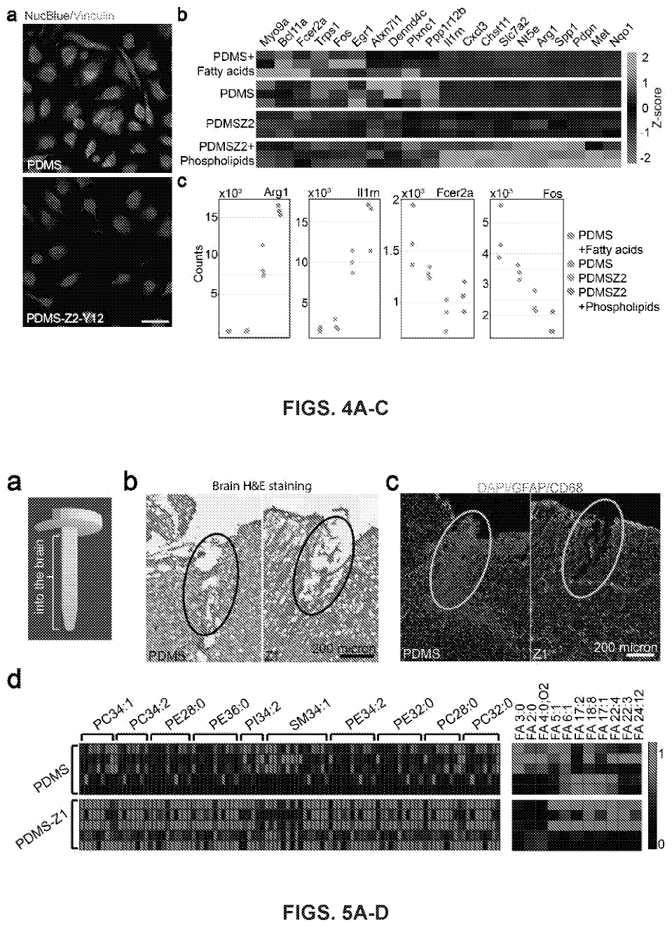

To further investigate immune cell interactions with the chemical-modified, anti-fibrotic implants, murine macrophages were cultured on Z2-functionalized and non-functionalized PDMS for 18 hours, and then immuno-stained for vinculin, and imaged via confocal microscopy. Vinculin is a cytoskeletal protein found on the cell membrane that is associated with cell adhesion to surfaces (Bays et al., 2017). Murine macrophages cultured on PDMS-Z2 exhibit less intense vinculin staining compared to cells on non-functionalized PDMS (FIG. 4a), indicating the Z2-Y12 may be weakening macrophage adhesion.

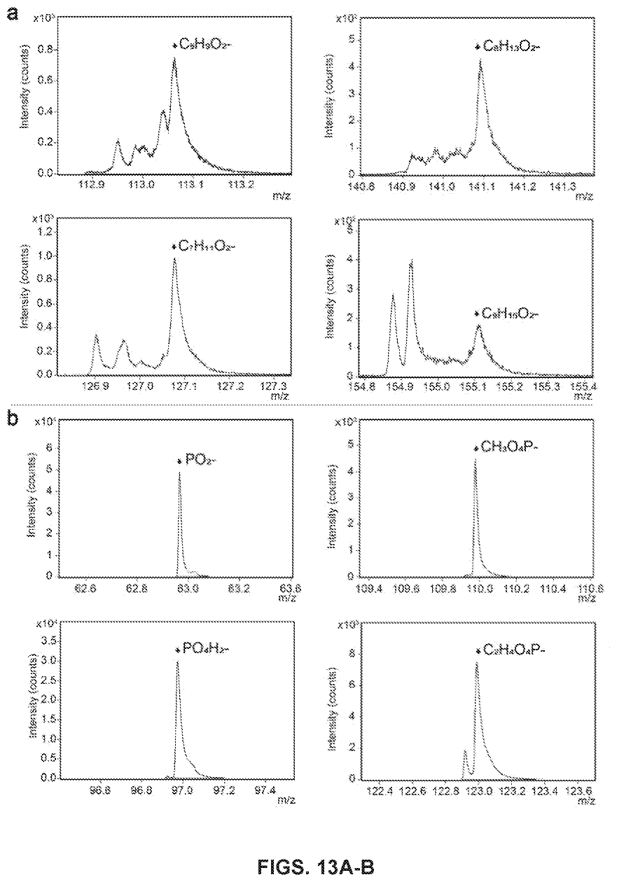

Lipids directly modulate the transcriptome of macrophages ex vivo. Now that the inventors determined that the anti-fibrotic small molecules affect the lipid deposition profiles and the transcriptome of immune cells in vivo, the inventors wanted to explore if the lipid profiles themselves affected how immune cells recognized the materials. To do this, bulk RNA sequencing analysis was performed on murine macrophages cultured on the PDMS disks in the absence or presence of PLs and FAs. The goal of this analysis was to determine the extent surface absorbed lipids can impact macrophage transcriptional profiles and polarization. Macrophages were extracted from the IP space of mice. To mimic the profiles that appear on the different implants, mixtures of FAs or PLs were spun coated onto unmodified and Z2-Y12-modified PDMS disks, respectively (FIGS. 13a-b). Macrophages were then cultured on the disks for 18 hours, after which their RNA was extracted and sequenced.

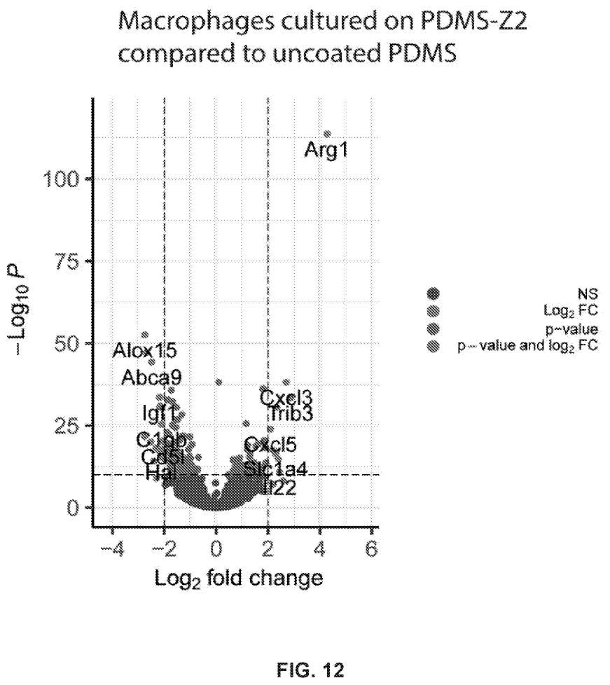

Macrophage transcriptional profiles were found to depend on both the surface functionalization of the PDMS disks and whether they were additionally coated with PLs or FA lipids (FIGS. 4b-c and FIG. 12). Hierarchical clustering analyses revealed two gene clusters that were either observed to be enriched when macrophages were cultured on non-functionalized or Z2 functionalized PDMS surfaces and further enriched in the presence of lipids (FIG. 4b). One cluster includes multiple genes that have been associated with pro-inflammatory responses (Fcer2a, Fos, Bcl11a, Dennd4c, and Plxnc1) (Kleinau et al., 1999; Zenz et al., 2008; Liu et al., 2020; Liu et al., 2006; Ho et al., 2016; Granja et al., 2014), which tended to be upregulated when macrophages were cultured directly on PDMS, with and without addition of FA lipids. The second cluster contains genes that have been associated with the anti-inflammatory responses (Arg1, Spp1, Nqo1, Met, Il1m, Slc7a2, Nt5e, Chst11, and Pdpn) (Kordab et al., 2018; Coburn et al., 2019; Fan et al., 2020; Arechederra et al., 2013; Isbir et al., 2008; Sun et al., 2021; Shin et al., 2007; Pourcet and Pineda-Torra, 2013; Desanti et al., 2018). These gene were expressed at low levels on the unmodified PDMS surfaces. The opposite response was observed for the expression of these gene clusters on the Z2-Y12 modified surfaces. The pro-inflammatory gene cluster was downregulated on the anti-fibrotic surface, while the immunomodulatory/immunosuppressive proteins were upregulated. Moreover, the addition of phospholipids appears to further enhance the expression of genes in the immunomodulatory cluster compared to expression patterns on the Z2-Y12 modified surface alone.

Four key immune response genes Arg1, Il-1m, Fcer2a, and Fos exhibited significant dependences on the Z2-Y12 chemical modification and lipid species absorbed to PDMS surfaces (FIG. 4c). As seen in the heatmaps, mRNA read counts were higher for Arg1 and Il-1m, key markers of inflammation suppression (Fan et al., 2020; Pesce et al., 2009), when macrophages were cultured on Z2 functionalized PDMS compared to non-functionalized PDMS. This response was potentiated further in the presence of PLs. In contrast, macrophages cultured on non-functionalized PDMS had higher expression of Fcer2a and Fos, two key pro-inflammatory protein markers (Zenz et al., 2008; Pforte et al., 1990). Their expression was potentiated further when FA lipids are also presented on PDMS disks (FIG. 4c). These results demonstrate synergistic effects where the immune response to a material is influenced by both the surface functionalization on and the lipids that associate with a material.

Surface modification governs lipid deposition profiles at multiple implant locations and with different materials. The inventors next examined the extent that the anti-fibrotic Z2 and Z1 surface functionalization influenced both the fibrotic properties of, and associated lipid composition profiles on implants placed at different anatomical locations. The impact of biomaterial location was assessed by implanting Z1 and non-functionalized pillars of PDMS (FIG. 5a) into the brain of mice for 2 weeks. Histology of the of brain tissue around the implant revealed distinct fibrotic strands of cells at the sites of non-functionalized PDMS implant, which were absent on the Z1 modified implants (FIG. 5b). Additionally, immune cells also appeared to accumulate at the PDMS implant site. This migration was not observed with the Z1 functionalized pillars (FIG. 5c), suggesting the Z1 small molecule convers immune-evasive properties. Phospholipid composition profiles on implants in mouse brains followed similar trends found with the PDMS disks implanted in the gonadal fat pads. Phospholipids were enriched significantly on the Z1 modified pillars compared to the non-functionalized implants (FIG. 5d). However, fatty acid enrichment was not observed on the non-functionalized implants. This difference could potentially be from the unique immune and lipid environment in the brain.

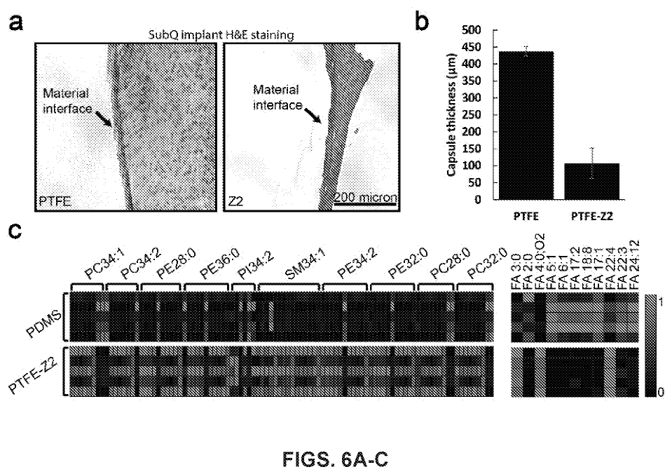

Finally, to assess the role of substrate material composition, the inventors evaluated lipid enrichment profiles on Z2 functionalized and non-functionalized disks composed of polytetrafluoroethylene (PTFE). PTFE disks were implanted in the subcutaneous space of mice. Histology of PTFE and PTFE-Z2-Y12 implants after 28 days in the murine subcutaneous space showed that the PTFE induce a thicker fibrotic capsule compared to PTFE-Z2-Y12 (FIGS. 6a-b). Moreover, phospholipid profiles measured by ToF-SIMs 24 hours after implantation closely mirrored the results found with PDMS-based implants placed in the gonadal fat pad or brain, with phospholipids being enriched on substrates that are functionalized with anti-fibrotic small molecules (FIG. 6c). FA profiles also matched that of the gonadal fat pad implants, with fatty acids being enriched on the fibrosis-inducing implants.

SUPPLEMENTARY TABLE 1

| PC34:1 | | PC34:2 | | PE28:0 | | PE36:0 | | P134:2 | |
|---|---|---|---|---|---|---|---|---|---|
| Mass | Species | Mass | Species | Mass | Species | Mass | Species | Mass | Species |
| 62.960 | PO₂— | 279.191 | C₁₃H₃₁O₂— | 140.075 | C₂H₇NO₄P— | 283.221 | C₁₃H₃₅O₂— | 241.044 | C₆H₁₀PO₃— |
| 78.954 | PO₃— | 62.960 | PO₂— | 122.017 | C₂H₅NO₃P— | 140.075 | C₂H₇NO₄P— | 299.011 | C₉H₁₆PO₉— |
| 96.979 | PO₄H₂— | 78.954 | PO₃— | 153.044 | C₃H₅NO₄P— | 122.017 | C₂H₅NO₃P— | 255.195 | C₁₆H₃₁O₂— |
| 109.960 | CH₃O₄P— | 96.979 | PO₄H₂— | 180.034 | C₅H₁₁NO₄P— | 153.044 | C₃H₅NO₄P— | 62.960 | PO₂— |
| 123.046 | C₂H₄O₄P— | 109.960 | CH₃O₄P— | 227.167 | C₁₄H₂₇O₂— | 180.034 | C₅H₁₁NO₄P— | 279.191 | C₁₃H₃₁O₂— |
| 158.905 | (null) | 123.046 | C₂H₄O₄P— | 62.960 | PO₂— | 227.167 | C₁₄H₂₇O₂— | 833.660 | C₄₃H₇₃PO₁₃— |
| 255.195 | C₁₆H₃₁O₂— | 158.905 | (null) | 78.954 | PO₃— | 62.960 | PO₂— | | |
| 281.205 | C₁₃H₃₃O₂— | 255.195 | C₁₆H₃₁O₂— | 96.979 | PO₄H₂— | 78.954 | PO₃— | | |
| | | | | 109.960 | CH₃O₄P— | 96.979 | PO₄H₂— | | |
| | | | | 158.905 | (null) | 109.960 | CH₃O₄P— | | |
| | | | | | | 158.905 | (null) | | |

Specific ion peaks from the ToF-SIMS reference library used to identify lipids. The species here correlate to the columns in the heatmap from FIG. 2d. Acronyms used are as follows: Phosphatidylinositol (PI), Phosphatidylethanolamine (PE), Phosphatidylcholine (PC), Sphingomyelin (SM). Cells underlined are considered “required peaks”, meaning they are more likely to exist than the rest of the peaks in the spectra of a sample containing the lipid the required peak belongs to. The order of species listed here matches the order of columns in the heatmap in FIG. 2d.

SUPPLEMENTARY TABLE 2

| SM34:1 | | PC34:2 | | PE32:0 | | PC28:0 | | PC32:0 | |
|---|---|---|---|---|---|---|---|---|---|
| Mass | Species | Mass | Species | Mass | Species | Mass | Species | Mass | Species |
| 687.442 | C₃₃H₇₆N₂O₆P— | 714.176 | C₃₉H₇₃NO₃P— | 690.502 | C₃₇H₇₃NO₃P— | 653.444 | (null) | 709.525 | (null) |
| 701.384 | C₃₉H₇₃N₂O₆P— | 140.075 | C₂H₇NO₄P— | 140.075 | C₂H₇NO₄P— | 62.960 | PO₂— | 62.960 | PO₂— |
| 642.492 | C₃₆H₆₉NO₆P— | 122.017 | C₂H₅NO₃P— | 122.017 | C₂H₅NO₃P— | 78.954 | PO₃— | 78.954 | PO₃— |
| 447.273 | C₂₂H₄₄N₂O₅P— | 153.044 | C₃H₃NO₄P— | 153.044 | C₃H₅NO₄P— | 96.979 | PO₄H₂— | 96.979 | PO₄H₂— |
| 402.224 | (null) | 180.034 | C₅H₁₁NO₄P— | 180.034 | C₅H₁₁NO₄P— | 109.960 | CH₃O₄P— | 109.960 | CH₃O₄P— |
| 616.459 | C₃₄H₆₇NO₆P— | 227.167 | C₁₄H₂₇O₂— | 227.167 | C₁₄H₂₇O₂— | 123.046 | C₂H₄O₄P— | 123.046 | C₂H₄O₄P— |
| 598.569 | C₃₄H₆₅NO₅P— | 62.960 | PO₂— | 62.960 | PO₂— | 158.905 | (null) | 158.905 | (null) |
| 404.280 | C₂₀H₃₉NO₅P— | 78.954 | PO₃— | 78.954 | PO₃— | 255.195 | C₁₆H₃₁O₂— | 255.195 | C₁₆H₃₁O₂— |
| 390.255 | (null) | 96.979 | PO₄H₂— | 96.979 | PO₄H₂— | 281.205 | C₁₈H₃₃O₂— | 281.205 | C₁₃H₃₃O₂— |
| 62.960 | PO₂— | 109.960 | CH₃O₄P— | 109.960 | CH₃O₄P— | | | | |
| 78.954 | PO₃— | 158.905 | (null) | 158.905 | (null) | | | | |
| 96.979 | PO₄H₂— | | | | | | | | |
| 109.960 | CH₃O₄P— | | | | | | | | |
| 123.046 | C₂H₄O₄P— | | | | | | | | |
| 158.905 | (null) | | | | | | | | |

Identified lipids from referencing ToF-SIMS data to LIPID MAPS. The species here correlate to the rows in the heatmap from FIG. 3a. FA stands for fatty acid.

### Example 3—Discussion

Fibrosis remains a dominant cause of implant rejection and failure that limits the efficacy of a wide range of medical devices. Methods to modify materials physically and chemically have been developed to mitigate implant-induced inflammation and the resultant deleterious growth of fibrotic tissue (Vegas et al., 2016a; Bridges et al., 2008)). The current design paradigm for engineering these immune-evasive materials is to prevent protein fouling and subsequent immune and fibroblast cell activation, adhesion and growth (Foggia et al., 2019; Klopfleisch and Jung, 2017: Vishwakarma et al., 2016; Chen et al., 2005; Sussman et al., 2014). This study uncovered that engineering implants to present immune-modulatory small-molecules can prevent fibrosis via a novel mechanism that likely involves regulation of the lipid profiles that are deposited their surfaces. This mechanism was discovered through a series of molecular, cellular and in vivo functional analyses. ToF-SIMS revealed that PC, PL, PE, and SM lipids preferentially deposit on immune-evasive biomaterials, while a set of 11 fatty acids were preferentially deposited on unmodified PDMS implants. Importantly, a non-functional molecular analog did not alter lipid deposition relative to control implants, implying that immune and attenuated fibrotic responses to the small molecules are not driven by physicochemical interactions, but rather by molecular/cellular recognition of the modified surface. Moreover, ToF-SIMS also demonstrated that lipid deposition patterns were consistent when implants were placed at multiple locations (brain, subcutaneous and IP space), for different implant materials (PDMS and PTFE) and in mice and humans.

Consistent with previous findings (Doloff et al., 2017; Veiseh et al., 2015), macrophages were a dominant immune cell type detected in scRNAseq experiments, indicated they are likely key orchestrators of the anti-fibrotic response. Moreover, the transcription of anti-inflammatory markers including Arg1 and Il1m were upregulated in macrophages in vitro when they were cultured on top of implants that are functionalized with Z2 and then coated with a mixture of phospholipids. These and other transcriptional changes indicate selective recruitment and polarization of suppressive macrophages at the surface of the implants. Importantly, the further potentiation of transcriptional changes by lipids on top changes induced by Z2 suggest there is a synergistic benefit to adding phospholipids to the molecular functionalized surfaces. Conversely, the addition of fatty acids exacerbated the inflammatory polarization of macrophages on biomaterial surfaces. The surface functionalization and surface lipid profiles in these in vitro analyses notably mirror those in the inventors' in vivo experiments. Overall, they demonstrate that the recruitment and polarization of inflammatory versus suppressive macrophages and resultant pro- versus non-fibrotic response can be driven not only via the surface presentation of immunomodulatory small molecules, but also by the molecular profiles of the lipids that associate with implant surfaces.

While the phospholipid and fatty acid profiles were robustly different across. functionalized and unmodified implants in multiple settings, the inventors were unable to detect changes in protein absorption via ToF-SIMs. However, this result does not rule out contributions due to changes in the composition or conformational states of proteins that can affect fibrotic response. Interestingly, lipid surface presentation has been shown to affect the proteins absorption (Zhdanov and Kasemo, 2010), and vice versa (Ben-Tal et al., 1996). This raises the possibility that the species of lipids and types of proteins that deposit initially on an implant may be inherently linked. Future studies could focus on exploring the interplay of proteins and lipids and their potential synergistic effects on fibrotic outcomes.

Here the inventors demonstrated that lipids play a role in the biomaterial-induced fibrotic response. They discovered that the lipid profile that deposits on an implant is dependent on the implant's immunogenicity, and that these lipids have a direct effect on the RNA profile of macrophages. Lastly, the inventors demonstrated that this lipid response is consistent across implant sites, materials, and species. The implications of these results are two-fold. From a fundamental science perspective, the inventors have expanded the knowledge of the molecular factors known to play a role in the biomaterial induced fibrotic response. From a biomedical engineering perspective, they have elucidated a novel mechanism in which the immunogenicity of a material can be modified to modulate FBR and fibrosis.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
L. W. McKeen, in Handbook of Polymer Applications in Medicine and Medical Devices, Elsevier Inc., 2014, pp. 21-53.
M. Saini, World Journal of Clinical Cases 2015, 3, 52.
B. Rolfe, J. Mooney, B. Zhang, S. Jahnke, S.-J. Le, Y.-Q. Chau, Q. Huang, H. Wang, G. Campbell, J. Campbell, Regenerative Medicine and Tissue Engineering—Cells and Biomaterials 2011, DOI 10.5772/21790.
T. Someya, Z. Bao, G. G. Malliaras, Nature 2016, 540, 379.
M. J. Foggia, R. V. Quevedo, M. R. Hansen, Laryngoscope Investigative Otolaryngology 2019, 4, 678.
R. Klopfleisch, F. Jung, The Pathology of the Foreign Body Reaction against Biomaterials, John Wiley And Sons Inc., 2017.
C. R. Jenney, J. M. Anderson, Journal of Biomedical Materials Research 2000, 49, 435.
A. Vishwakarma, N. S. Bhise, M. B. Evangelista, J. Rouwkema, M. R. Dokmeci, A. M. Ghaemmaghami, N. E. Vrana, A. Khademhosseini, Trends in Biotechnology 2016, 34, 470.
J. C. Doloff, O. Veiseh, R. de Mezerville, M. Sforza, T. A. Perry, J. Haupt, M. Jamiel, C. Chambers, A. Nash, S. Aghlara-Fotovat, J. L. Stelzel, S. J. Bauer, S. Y. Neshat, J. Hancock, N. A. Romero, Y. E. Hidalgo, I. M. Leiva, A. M. Munhoz, A. Bayat, B. M. Kinney, H. C. Hodges, R. N. Miranda, M. W. Clemens, R. Langer, Nature Biomedical Engineering 2021, 5, 1115.
N. Noskovicova, R. Schuster, S. van Putten, M. Ezzo, A. Koehler, S. Boo, N. M. Coelho, D. Griggs, P. Ruminski, C. A. McCulloch, B. Hinz, Nature Biomedical Engineering 2021, 5, 1437.
V. Figueroa, C. Milla, E. J. Parks, S. J. Schwarzenberg, A. Moran, The American Journal of Clinical Nutrition 2002, 75, 1005.
V. Chiurchiù, A. Leuti, M. Maccarrone, Frontiers in Immunology 2018, 9, DOI 10.3389/fimmu.2018.00038.
C. J. Binder, N. Papac-Milicevic, J. L. Witztum, Nature Reviews Immunology 2016, 16, 485.
G. S. Hotamisligil, Nature 2017 2017, 542, 177.
A. W. Bridges, A. J. García, Journal of Diabetes Science and Technology 2008, 2, 984.
S. Chen, J. Zheng, L. Li, S. Jiang, J Am Chem Soc 2005, 127, 14473.
E. M. Sussman, M. C. Halpin, J. Muster, R. T. Moon, B. D. Ratner, Annals of Biomedical Engineering 2014, 42, 1508.
A. J. Vegas, O. Veiseh, J. C. Doloff, M. Ma, H. H. Tam, K. Bratlie, J. Li, A. R. Bader, E. Langan, K. Olejnik, P. Fenton, J. W. Kang, J. Hollister-Locke, M. A. Bochenek, A. Chiu, S. Siebert, K. Tang, S. Jhunjhunwala, S. Aresta-Dasilva, N. Dholakia, R. Thakrar, T. Vietti, M. Chen, J. Cohen, K. Siniakowicz, M. Qi, J. McGarrigle, A. C. Graham, S. Lyle, D. M. Harlan, D. L. Greiner, J. Oberholzer, G. C. Weir, R. Langer, D. G. Anderson, Nature Biotechnology 2016a, 34, 345.
A. J. Vegas, O. Veiseh, M. Gürtler, J. R. Millman, F. W. Pagliuca, A. R. Bader, J. C. Doloff, J. Li, M. Chen, K. Olejnik, H. H. Tam, S. Jhunjhunwala, E. Langan, S. Aresta-Dasilva, S. Gandham, J. J. McGarrigle, M. A. Bochenek, J. Hollister-Lock, J. Oberholzer, D. L. Greiner, G. C. Weir, D. A. Melton, R. Langer, D. G. Anderson, Nature Medicine 2016b, 22, 306.
X. Xie, J. C. Doloff, V. Yesilyurt, A. Sadraei, J. J. McGarrigle, M. Omami, O. Veiseh, S. Farah, D. Isa, S. Ghani, I. Joshi, A. Vegas, J. Li, W. Wang, A. Bader, H. H. Tam, J. Tao, H. J. Chen, B. Yang, K. A. Williamson, J. Oberholzer, R. Langer, D. G. Anderson, Nature Biomedical Engineering 2018, 2, 894.
S. Bose, L. R. Volpatti, D. Thiono, V. Yesilyurt, C. McGladrigan, Y. Tang, A. Facklam, A. Wang, S. Jhunjhunwala, O. Veiseh, J. Hollister-Lock, C. Bhattacharya, G. C. Weir, D. L. Greiner, R. Langer, D. G. Anderson, Nature Biomedical Engineering 2020, 4, 814.
R. K. Ghanta, S. Aghlara-Fotovat, A. Pugazenthi, C. T. Ryan, V. P. Singh, M. Mathison, M. I. Jarvis, S. Mukherjee, A. Hernandez, O. Veiseh, Biomaterials Science 2020, 8, 5061.
M. A. Bochenek, O. Veiseh, A. J. Vegas, J. J. McGarrigle, M. Qi, E. Marchese, M. Omami, J. C. Doloff, J. Mendoza-Elias, M. Nourmohammadzadeh, A. Khan, C.-C. C. Yeh, Y. Xing, D. Isa, S. Ghani, J. Li, C. Landry, A. R. Bader, K. Olejnik, M. Chen, J. Hollister-Lock, Y. Wang, D. L. Greiner, G. C. Weir, B. L. Strand, A. M. A. Rokstad, I. Lacik, R. Langer, D. G. Anderson, J. Oberholzer, Nature Biomedical Engineering 2018, 2, 810.
“Safety & Efficacy of Encapsulated Allogeneic FVIII Cell Therapy in Haemophilia A—clinicaltrials.gov/ct2/show/NCT04541628?term=Sigilon&draw=2&rank=1, 2020.
B. Y. Yoo, B. H. Kim, J. S. Lee, B. H. Shin, H. Kwon, W. G. Koh, C. Y. Heo, Acta Biomaterialia 2018, 76, 56.
K. W. Meacham, R. J. Giuly, L. Guo, S. Hochman, S. P. DeWeerth, Biomedical Microdevices 2008, 10, 259.
A. Mata, A. J. Fleischman, S. Roy, Biomedical Microdevices 2005, 7, 281.
J. C. Doloff, O. Veiseh, A. J. Vegas, H. H. Tam, S. Farah, M. Ma, J. Li, A. Bader, A. Chiu, A. Sadraei, S. Aresta-Dasilva, M. Griffin, S. Jhunjhunwala, M. Webber, S. Siebert, K. Tang, M. Chen, E. Langan, N. Dholokia, R. Thakrar, M. Qi, J. Oberholzer, D. L. Greiner, R. Langer, D. G. Anderson, Nature Materials 2017, 16, 671.
M. J. Taylor, K. Y. Zhang, D. J. Graham, L. J. Gamble, Surface Science Spectra 2018, 25, 025001.
B. Johansson, Surface and Interface Analysis 2006, 38, 1401.
M. K. Passarelli, N. Winograd, Biochimica et Biophysica Acta—Molecular and Cell Biology of Lipids 2011, 1811, 976.
S. H. Zhang, R. L. Reddick, J. A. Piedrahita, N. Maeda, Science 1992, 258, 468.
G. Lo Sasso, W. K. Schlage, S. Boué, E. Veljkovic, M. C. Peitsch, J. Hoeng, Journal of Translational Medicine 2016, 14, 146.
J. L. Bays, K. A. DeMali, Cellular and Molecular Life Sciences 2017, 74, 2999.
[S. Kleinau, P. Martinsson, S. Gustavsson, B. Heyman, The Journal of Immunology 1999, 162.
R. Zenz, R. Eferl, C. Scheinecker, K. Redlich, J. Smolen, H. B. Schonthaler, L. Kenner, E. Tschachler, E. F. Wagner, Arthritis Research and Therapy 2008, 10, DOI 10.1186/ar2338.
X. Liu, L. Yang, D. Jiang, W. Lu, Y. Zhang, Cancer Cell International 2020, 20, DOI 10.1186/s12935-020-01346-0.
H. Liu, G. C. Ippolito, J. K. Wall, T. Niu, L. Probst, B. S. Lee, K. Pulford, A. H. Banham, L. Stockwin, A. L. Shaffer, L. M. Staudt, C. Das, M. J. S. Dyer, P. W. Tucker, Molecular Cancer 2006, 5, 1.
L. C. Ho, J. M. Sung, Y. T. Shen, H. F. Jheng, S. H. Chen, P. J. Tsai, Y. S. Tsai, Journal of Molecular Medicine 2016, 94, 933.
T. Granja, D. Kohler, V. Mirakaj, E. Nelson, K. Konig, P. Rosenberger, Mucosal Immunology 2014, 7, 879.
T. Kordab, W. Osen, S. B. Eichmüller, Frontiers in Immunology 2018, 9, DOI 10.3389/fimmu.2018.00813.
L. A. Coburn, K. Singh, M. Asim, D. P. Barry, M. M. Allaman, N. T. Al-Greene, D. M. Hardbower, D. Polosukhina, C. S. Williams, A. G. Delgado, M. B. Piazuelo, M. K. Washington, A. P. Gobert, K. T. Wilson, Oncogene 2019, 38, 1067.
Y. C. Fan, W. Y. Chen, K. Der Lee, Y. C. Tsai, Biology (Basel) 2020, 9, DOI 10.3390/biology9040067.
M. Arechederra, R. Carmona, M. González-Nuñez, Á. Gutiérrez-Uzquiza, P. Bragado, I. Cruz-González, E. Cano, C. Guerrero, A. Sánchez, J. M. López-Novoa, M. D. Schneider, F. Maina, R. Muñoz-Chápuli, A. Porras, Biochimica et BiophysicaActa—Molecular Basis of Disease 2013, 1832, 2204.
C. S. Isbir, A. Ergen, A. Tekeli, U. Zeybek, U. Gormus, S. Arsan, Cell Biochemistry and Function 2008, 26, 534.
X. Sun, X. He, Y. Zhang, K. Hosaka, P. Andersson, J. Wu, J. Wu, X. Jing, Q. Du, X. Hui, B. Ding, Z. Guo, A. Hong, X. Liu, Y. Wang, Q. Ji, R. Beyaert, Y. Yang, Q. Li, Y. Cao, Gut 2021, 71, 129.
H. D. Shin, B. L. Park, H. S. Cheong, J. H. Yoon, Y. J. Kim, H. S. Lee, International Journal of Epidemiology 2007, 36, 1001.
B. Pourcet, I. Pineda-Torra, Trends in Cardiovascular Medicine 2013, 23, 143.
G. Desanti, A. Saghir, A. Naylor, S. Kemble, J. Falconer, C. Wehmeyer, J. Marshall, K. Nakamura, M. Goodall, L. Navarro-Núñez, S. Watson, C. Buckley, in Annals of the Rheumatic Diseases, BMJ, 2018, p. A7.2-A8.
J. T. Pesce, T. R. Ramalingam, M. M. Mentink-Kane, M. S. Wilson, K. C. E. Kasmi, A. M. Smith, R. W. Thompson, A. W. Cheever, P. J. Murray, T. A. Wynn, PLoS Pathogens 2009, 5, DOI 10.1371/journal.ppat.1000371.
A. Pforte, G. Breyer, J. C. Prinz, P. Gais, G. Burger, K. Häussinger, E. P. Rieber, E. Held, H. W. L. Ziegler-Heitbrock, Journal of Experimental Medicine 1990, 171, 1163.
A. W. Bridges, N. Singh, K. L. Burns, J. E. Babensee, L. Andrew Lyon, A. J. García, Biomaterials 2008, 29, 4605.
O. Veiseh, J. C. Doloff, M. Ma, A. J. Vegas, H. H. Tam, A. R. Bader, J. Li, E. Langan, J. Wyckoff, W. S. Loo, S. Jhunjhunwala, A. Chiu, S. Siebert, K. Tang, J. Hollister-Lock, S. Aresta-Dasilva, M. Bochenek, J. Mendoza-Elias, Y. Wang, M. Qi, D. M. Lavin, M. Chen, N. Dholakia, R. Thakrar, I. Lacik, G. C. Weir, J. Oberholzer, D. L. Greiner, R. Langer, D. G. Anderson, Nature Materials 2015, 14, 643.
V. P. Zhdanov, B. Kasemo, Biophysical Chemistry 2010, 146, 60.
N. Ben-Tal, B. Honig, R. M. Peitzsch, G. Denisov, S. McLaughlin, Biophysical Journal 1996, 71, 561.
M. Gabriel, K. Niederer, H. Frey, Journal of Visualized Experiments: JoVE 2016, 2016, 54272.
Y. Hao, S. Hao, E. Andersen-Nissen, W. M. Mauck, S. Zheng, A. Butler, M. J. Lee, A. J. Wilk, C. Darby, M. Zager, P. Hoffman, M. Stoeckius, E. Papalexi, E. P. Mimitou, J. Jain, A. Srivastava, T. Stuart, L. M. Fleming, B. Yeung, A. J. Rogers, J. M. McElrath, C. A. Blish, R. Gottardo, P. Smibert, R. Satija, Cell 2021, 184, 3573.
A. M. Bolger, M. Lohse, B. Usadel, Bioinformatics 2014, 30, 2114.
N. L. Bray, H. Pimentel, P. Melsted, L. Pachter, Nature Biotechnology 2016, 34, 525.
H. Pimentel, N. L. Bray, S. Puente, P. Melsted, L. Pachter, Nature Methods 2017, 14, 687.
C. Soneson, M. I Love, M. D. Robinson, F1000Res 2015, 4, 1521.
M. I. Love, W. Huber, S. Anders, Genome Biology 2014, 15, DOI 10.1186/s13059-014-0550-8.

1. A method of identifying a lipid having immune regulating properties comprising:
(a) providing
(i) a surface coated with one or more small molecules that are immune-evasive; or
(ii) an uncoated surface or a surface coated with one or more small molecules that are immune-evoking;
(b) introducing said surface into a living animal;
(c) removing said surface after a period of time sufficient for lipids to interact with said surface; and
(d) identifying a lipid deposited on the surface of said coating.

2. The method of claim 1, wherein said surface is a flat surface (e.g., a disc), a curved surface, such a sphere, the interior or exterior of a tube (e.g., a catheter), on a bead, or on a rod.

3. The method of claim 1, wherein the surface comprises an organic polymer, such as polydimethylsiloxane (PDMS), or a hydrogel, such as an alginate.

4. The method of claim 1, wherein the one or more small molecules that are immune-evasive comprise Z1-Y12 and/or Z1-Y15.

5. The method of claim 1, wherein the uncoated surface comprises an organic polymer, such as polydimethylsiloxane (PDMS) or a hydrogel, such as an alginate.

6. The method of claim 1, wherein the one or more small molecules that are immune-evoking comprise Z1-Y12A.

7. The method of claim 1, wherein the period of time in step (c) is about 24 hours or more, such as 1 day, 2 days, 3, days, 4, days, 5, days, 6, days, 7 days, 10 days, two weeks, three weeks or four weeks.

8. The method of claim 1, wherein step (d) comprises mass spectrometry (e.g., Time of Flight-Secondary Ion Mass Spectrometry (ToF-SIMS),) chromatography (e.g., thin layer chromatography, gas chromatography, high pressure liquid chromatography), nuclear magnetic resonance spectroscopy, or any combination thereof.

9. The method of claim 1, wherein said animal is a human or non-human mammal.

10. The method of claim 1, wherein said surface is implanted subcutaneously, inserted into a body orifice, implanted intraperitoneally, or implanted into the brain.

11. A medical device coated with:
(a) one or more immune-evasive lipids; or
(b) one or more immune-evoking lipids,
wherein one or more immune-evasive lipids comprise a phospholipid, or wherein one or more immune-evoking lipids comprise a fatty acid.

12. The medical device of claim 11, wherein the phospholipid is selected from phosphatidyl choline, phosphatidyl ethanolamine, phosphatidyl inositol and/or sphingomyelin.

13. The medical device of claim 12, comprising two or more of said phospholipids.

14. The medical device of claim 11, wherein the fatty acid is selected from the fatty acids of Table 2.

15. The medical device of claim 14, comprising two or more of said fatty acids.

16. The medical device of claim 11, wherein the medical device is an implantable device, a cardiac pacemaker, a catheter, a needle injection catheter, a blood clot filter, a vascular transplant, a balloon, a stent transplant, a biliary stent, an intestinal stent, a bronchial stent, an esophageal stent, a ureteral stent, an aneurysm-filling coil or other coil device, a surgical repair mesh, a breast implant, a silicone implant, a transmyocardial revascularization device, a percutaneous myocardial revascularization device, a prosthesis, an artificial organ, an artificial vessel, a tube, an organ replacement part, a fiber, a hollow fiber, a membrane, a textile, a dialyzer, a connecting piece, a sensor, a valve, an endoscope, a filter, or a pump chamber.

17. The medical device of claim 11, wherein said medical device is comprised of an organic polymer, such as polydimethylsiloxane (PDMS), or a hydrogel, such as an alginate.

18. The medical device of claim 11, wherein said medical device further comprises other immune-evasive modifications including non-fouling hydrophilic polymeric film coatings, semi-permeable hydrogel coatings, surface modification with immunomodulatory proteins, surface topography/geometry modifications, immunomodulating cells, surface charge modifications, and combination thereof.

19. The medical device of claim 11, wherein said medical device has multiple layers of immune-evasive lipids and/or immune-evoking lipids.

20. A method of treating a disease or disorder comprising implanting a medical device according to claim 11 into a subject in need thereof.

21. The method of claim 20, wherein said medical device is co-delivered with and immune-modulatory small molecule drug or biologic.
